SEQUENCE LISTING (2 sequences)

SEQ ID NO: 1 - protein, 1001 aa, Drosophila melanogaster
MSPNRWILLLIFYISYLMFGAAIYYHIEHGEEKISRAEQRKAQIAINEYLLEELGDKNTTTQDEILQRISDYCDKPVTLPPTYDDTPYTWTFYHAFFFAFTVCSTVGYGNISPTTFAGRMIMIAYSVIGIPVNGILFAGLGEYFGRTFEAIYRRYKKYKMSTDMHYVPPQLGLITTVVIALIPGIALFLLLPSWVFTYFENWPYSISLYYSYVTTTTIGFGDYVPTFGANQPKEFGGWFVVYQIFVIVWFIFSLGYLVMIMTFITRGLQSKKLAYLEQQLSSNLKATQNRIWSGVTKDVGYLRRMLNELYILKVKPVYTDVDIAYTLPRSNSCPDLSMYRVEPAPIPSRKRAFSVCADMVAAQREAGMVHANSDTELSKLDREKTFETAEAYRQTTDLLAKVVNALATVKPPPAEQEDAALYGGYHGFSDSQILASEWSFSTVNEFTSPRRPRARACSDFNLEAPRWQSERPLRSSHNEWTWSGDNQQIQEAFNQRYKGQQRANGAANSTMVHLEPDALEEQLKKQSPGAGRVKKFSMPDGLRRLFPFQKKRPSQDLERKLSVVSVPEGVISQQARSPLDYYSNTVTAASSQSYLRNGRGPPPPFESNGSLASGGGGLTNMGFQMEDGATPPSALGGGAYQRKAAAGKRRRESIYTQNQAPSARRGSMYPPTAHALAQMQMRRGSLATSGSGSAAMAAVAARRGSLFPATASASSLTSAPRRSSIFSVTSEKDMNVLEQTTIADLIRALEVVHTHAVLDEQQQAAAAGGAAGGGGISRGSRKQRKMGNAGLEPPQLPPILSLFAGDQTRTLQAAAANRLYARRSTIVGISPTGGAATAPAARSLLEPPPSYTERAANQSQITAGPSNAPTVQSKFRRRFSVRPTALQIPPGQAPPPGASLMEQSSQTALQRRLSLRPSPLARELSPTSPPGGSGSALPAGAIDESGGTSAQRLLPLPAGTRPSTSSTHSPLSRIVQISQAQRKSSMPSAAATGSSGAPAEK

SEQ ID NO: 2 - protein, 411 aa, Homo sapiens
MAAPDLLDPKSAAQNSKPRLSFSTKPTVLASRVESDTTINVMKWKTVSTIFLVVVLYLIIGATVFKALEQPHEISQRTTIVIQKQTFISQHSCVNSTELDELIQQIVAAINAGIIPLGNTSNQISHWDLGSSFFFAGTVITTIGFGNISPRTEGGKIFCIIYALLGIPLFGFLLAGVGDQLGTIFGKGIAKVEDTFIKWNVSQTKIRIISTIIFILFGCVLFVALPAIIFKHIEGWSALDAIYFVVITLTTIGFGDYVAGGSDIEYLDFYKPVVWFWILVGLAYFAAVLSMIGDWLRVISKKTKEEVGEFRAHAAEWTANVTAEFKETRRRLSVEIYDKFQRATSIKRKLSAELAGNHNQELTPCRRTLSVNHLTSERDVLPPLLKTESIYLNGLTPHCAGEEIAVIENIK

(19) United States
(12) Patent Application Publication
(10) Pub. No.: US 2004/0143855 A1
(43) Pub. Date: Jul. 22, 2004

(54) Ion channels as targets for sleep-related drugs

(76) Inventors: Giulio Tononi, Verona, WI (US); Chiara Cirelli, Verona, WI (US)

Correspondence Address: GODFREY & KAHN, S.C., 780 N. WATER STREET, MILWAUKEE, WI, 53202, US

(21) Appl. No.: 10/745,210

(22) Filed: Dec. 23, 2003

Related U.S. Application Data
(60) Provisional application No. 60/436,201, filed on Dec. 23, 2002.

(51) Int. Cl. A01K 67/033

(52) U.S. Cl. 800/8

(57) ABSTRACT

The invention provides screening methods for isolating short sleep, no rebound and sleep deprivation resistant Drosophila mutants. Such mutants are further useful to facilitate identification of sleep-related molecular targets. The invention also encompasses methods for identifying a sleep or wakefulness-promoting compound based on the compound's ability to modulate two pore domain K+ channels.

### CROSS-REFERENCE TO RELATED APPLICATION

This application claims the benefit of U.S. Provisional Application No. 60/436,201, filed Dec. 23, 2002, incorporated herein by reference.

### FIELD OF THE INVENTION

This invention relates generally to sleep-related drugs. In particular, the invention is directed to methods for identifying: (1) sleep-related Drosophila mutants; (2) sleep-related molecular targets; and (3) drugs which promote wakefulness or highly restorative sleep.

### BACKGROUND OF THE INVENTION

Sleep is one of the greatest unsolved mysteries of science today. No one knows why we sleep. That is, the physiological function fulfilled by sleep has not been identified. Sleep is homeostatically regulated and, in general, the longer an animal is awake, the more it needs to sleep.

It is well known that total sleep deprivation for more than a few hours affects cognitive performance (Doran et al., 2001). It is also known that even a limited sleep restriction, if prolonged for several days, significantly impairs cognitive performance (Dinges et al., 1997; Van Dongen et al., 2003). Sleep deprived subjects tend to take longer to respond to stimuli, particularly when tasks are monotonous and low in cognitive demands. However, sleep deprivation produces more than just decreased alertness. It is now evident that also tasks emphasizing higher cognitive functions, such as logical reasoning, encoding, decoding, and parsing complex sentences, complex subtraction tasks, and tasks requiring divergent thinking, such as those involving a flexible thinking style and the ability to focus on a large number of goals simultaneously, are significantly affected even after one night of sleep deprivation. Finally, tasks requiring sustained attention, such as those including goal-directed activities, can also be impaired by even a few hours of sleep loss. Thus, sleep loss causes attention deficits, decrease in short-term memory, speech impediments, and inflexible thinking (Harrison and Home, 2000).

Recent studies indicate that lack of sleep affects cognitive functions by destabilizing performance, rather than by eliminating completely the capacity to perform. Thus, a sleepy driver will either respond normally to an emergency or not at all, due to rapid changes in vigilance state. As a consequence, subjects may still be able to transiently perform at baseline levels in short tests even after 3-4 days of sleep deprivation. However, the same subjects will perform very poorly when engaged in tasks requiring sustained attention (Doran et al., 2001).

Previous attempts to use behavioral or pharmacological approaches in humans to preserve sustained cognitive performance without sleep have largely failed. For example, all available waking-promoting drugs have side effects, and none is effective after sustained periods of sleep deprivation (more than 10 hours; see e.g. Pigeau et al., 1995). Amphetamine effects depend on enhanced release of monoamine, blockade of the dopamine transporter, and inhibition of monoamino oxidases. Amphetamines have significant abuse potential, cause behavioral excitation, loss of appetite, increase in heart rate and blood pressure, and sleep rebound. Xanthine derivatives such as caffeine have fewer side effects, but also reduced potency. New drugs include modafinil, whose action does not appear to depend on newly synthesized endogenous catecholamines. Modafinil does not cause behavioral excitation nor sleep rebound, and it has been successful used to counteract excessive daytime sleepiness in narcolepsy and Parkinson's disease. However, in a recent study that measured aviator performance after one night of sleep deprivation, modafinil was found to be only partially effective in counterbalancing the cognitive effects of sleep deprivation (Caldwell et al., 2000). In another 64-h sleep deprivation study, both amphetamines and modafinil were able to fully counteract the cognitive deficits associated with sleep loss only for the first 10 hours of sleep deprivation (Pigeau et al., 1995).

Similarly, none of the hypnotics currently available are able to mimic all the physiological aspects of sleep and to concentrate them in a short and yet highly restorative “power nap”. Classical benzodiazepines such as zolpidem and zopiclone induce a non-physiological, non-restorative sleep since, among other effects, they decrease slow wave activity. Newer compounds such a gaboxadol that, unlike benzodiazepines, act as selective GABA_{A} receptor agonists, can increase slow wave activity (Faulhaber et al., 1997; Lancel et al., 2001). However, the slow oscillations of physiological slow wave sleep are characterized not by increased GABAergic inhibition, but rather by disfacilitation due to increased leak conductances (Steriade et al., 2001). Moreover, gaboxadol, far from reducing the amount of sleep needed for restoring performance, prolongs total sleep time (Faulhaber et al., 1997; Lancel et al., 2001).

The main reason for the present lack of more powerful and specific vigilance promoting drugs is that we still have little understanding of why sleep is needed to restore normal performance. Indeed, to date the only effective method to recover from the cognitive deficits caused by sleep loss is to be able to sleep. Furthermore, several studies suggest that not just few hours of sleep, but several days of normal sleep/waking patterns are required to normalize cognitive performance after sleep deprivation (Dinges et al., 1997).

As can be appreciated, currently available drugs used to modulate vigilance such as drugs that induce sleep, prolong wakefulness, or enhance alertness suffer from a number of shortcomings. Available sleep inducing drugs do not achieve the fully restorative effects of normal sleep and can cause undesirable effects upon waking such as anxiety or continued sedation. As well, many available drugs that promote wakefulness do so with a characteristic crash when the effect of the drugs wears off. Furthermore, many of the currently available drugs that modulate sleep and wakefulness are addictive or have adverse effects on learning and memory. More specific and effective drugs would clearly fill a societal need. Clearly, there exists a need to develop methods for identifying useful drugs that induce restorative sleep or that increase vigilance without undesirable side affects.

### SUMMARY OF THE INVENTION

In certain embodiments, the invention encompasses unique methods of identifying Drosophila melanogaster lines which exhibit short sleep, no rebound, or sleep deprivation resistant phenotypes. Mutant fly lines identified by the present invention consequently facilitate the rapid identification of molecular targets useful for sleep-related drug screening.

In yet other embodiments of the invention, methods of identifying new categories of sleep-related drugs capable of promoting wakefulness and inducing/maintaining sleep in a manner fundamentally different from available stimulants/hypnotics are provided. Specifically, the methods facilitate identification of compounds promoting wakefulness which act by decreasing or blocking K+ current through two pore domain K+ channels. Alternatively, sleep-promoting drugs are identified by their ability to increase K+ current through two pore domain K+ channels. In preferred embodiments, screening for sleep-related drugs is performed using the two pore domain K+ channel TREK-1 or a homolog thereof. Additional two pore domain K+ channels useful in practicing the present invention include ORK-1 or a homolog thereof.

The invention is also directed to methods of identifying sleep-related drugs which include the steps of: (a) administering a test compound to a test subject, preferably a wild type (wt) Drosophila fly; and (b) recording sleep quantity and vigilance of the wt fly over a time course including a baseline period, a sleep deprivation period, and a recovery period. Wakefulness-promoting drugs are identified as those bestowing upon test subjects improved wakefulness during the recovery period as compared to wild type Drosophila not receiving the test compound and subjected to the same time course. Continuous performance drugs are identified as those bestowing upon test subjects during the recovery period improved vigilance as compared to wt Drosophila not receiving the test compound and subjected to the same time course.

Other objects, features and advantages of the present invention will become apparent after review of the specification, claims and drawings.

### BRIEF DESCRIPTION OF THE DRAWINGS

FIG. 1. Analysis of locomotor activity and sleep in fruit flies. A. Schematic of the ultrasound activity monitoring system. A 44-kHz standing wave is passed across an independent enclosure containing a single fly. An integrated circuit samples a portion of each wave as a function of the transmit signal and compares it to the output from the receive signal for the same time window. When the fly moves its mass within the field, it perturbs the standing wave, and the resulting difference is counted as a movement. The output is sampled by a PC at 200 Hz, data are summed in 2-s bins, and stored for later processing (based on Shaw et al., 2000b) B. Twenty-four hour locomotor activity of a single female wild-type Canton-S fly as measured by the DAMS infrared system, as described herein. The fly is mostly active during the light period (from 8 am to 8 pm), and inactive during the dark period, when episodes of uninterrupted quiescence can last for several hours. C. Typical pattern of sleep in a population of 96 female wild-type Canton-S flies as measured in a DAMS monitor. DAMS measures activity as counts (number of crossings) per minute. Wakefulness is defined as any period of at least 1 minute characterized by activity (one or more counts per minute; see B). Based on arousal threshold data, sleep is defined as any period of uninterrupted behavioral quiescence (no counts/min) lasting for at least 5 min. Mean values of the amount of sleep are calculated for consecutive 30-min time intervals and the time course is graphically shown over the entire day. In female flies, most of the sleep occurs at night.

FIG. 2. The homeostatic regulation of sleep in the fruit flies. A. The increase in sleep duration following 6, 12, and 24 hours of sleep deprivation (SD) in female wild-type Canton-S flies (n=20-40 for each experiment). Each diagram shows the daily amount of sleep for baseline day (line 10), SD day (line 12), and the first recovery day after SD (line 14). Time and duration of SD are indicated by the bars below the x axis. An increase in sleep duration is present after all 3 periods of SD, and occurs mainly during the first 6 hours following the end of SD. Flies were maintained in a 12:12 light dark cycle (light on at 8 am). B. Amount of sleep lost (during SD) and of sleep recovered (during the first 6 hours of recovery day 1) for the experiments shown in A. More sleep is recovered after 12-24 h SD than after 6 h SD. Since female flies sleep mostly during the night, there is no significant difference in the amount of sleep lost and sleep recovered between 24 h SD and 12 h SD during the night. However, when female flies are sleep deprived for 12 hours during the light period (the corresponding graph is not shown in A), there is no significant sleep loss, nor significant sleep rebound. Note that the upper and lower scales on the y axis are different. Note also that in flies, as in mammals, the amount of sleep recovered after SD represents only a fraction of the sleep lost. C. To measure sleep fragmentation, a sleep continuity score is calculated, which increases during continuous epochs with no locomotor activity and decreases during epochs with one or more counts of activity. The sleep continuity score is high if sleep is continuous and undisturbed, and low if sleep is fragmented. Lines in the upper diagram represent sleep scores for 16 individual female Canton-S wild-type flies during baseline. In the lower diagram additional lines show the sleep score for the same flies the day following 24 h SD. Note the significant increase in the sleep score immediately after the end of SD.

FIG. 3. Identification of short sleep mutant lines. A. Intra-individual consistency and inter-individual variability in the daily amount of sleep in fruit flies. Daily amount of sleep is shown for four 7-day old virgin female flies of the same mutant line. B. Daily amount of sleep in 1547 insertional lines (P lines, female flies). Mean amount of sleep/24 hour is 616±169 (mean ±SD; min 131, max 1155). Shaded areas show one and two standard deviations from the mean. C. Daily amount of sleep in female (upper panel, n=16) and male (lower panel, n=15) flies of a short sleeper line. For comparison, the line 20 in each panel represents the daily amount of sleep in wild-type Canton-S flies (n=16).

FIG. 4. Identification of no rebound mutant lines. A. Cumulative graph showing the time course of the sleep rebound following 24 hour of sleep deprivation (SD) in female wild-type Canton-S flies. Daily amount of sleep during baseline was 580 min. Sleep recovered is expressed as % of sleep lost. At the end of recovery day 1, ˜40% of sleep was recovered, half of which during the first 2-3 hours following the end of SD (circle). No further recovery occurred during recovery day 2. B. Percentage of sleep recovered during the first 6 hours following 24 h SD in 593 insertional lines (P lines, female flies). Most lines recovered ˜20% of the sleep lost during SD. C. Increase in the sleep continuity score after 24 h SD in 593 insertional lines (same lines as in B). Bars indicate sleep scores for the first 6 hours of the light period during baseline (sleep score=37±30, mean ±SD, line 21) and after 24 h SD (118±67, line 23). The higher the sleep score, the lower the sleep fragmentation.

FIG. 5. A. A schematic of one heating chamber useful in carrying out a vigilance assay. The chamber 30 includes upper and lower surfaces 32a, 32b consisting of Peltier elements. Chamber 30 further includes sides 34a, 34b manufactured from acrylic glass plates. A control circuit comprising a computer 36, a power supply 38 and a thermosensor 40 at the chamber keep the Peltier elements at a permissive cold or restrictive hot temperature. Chamber 30 is virtually subdivided perpendicular to its long axis into two halves (left and right) by a directionally selective light gate 42 that informs the computer 36 about whether the fly is in the left or right half of the chamber. Light gate 42 consists of 2 infrared-emitting diodes and 2 infrared-sensitive photodetectors (not shown).

FIG. 6. A. A mating scheme for isolation of recessive X-linked mutations. B. A mating scheme for isolation of recessive autosomal mutations.

FIG. 7. Diagram illustrating the duration and distribution of sleep episodes for 13 flies is shown across a 24 hour period.

FIG. 8. Effects of sleep deprivation (SD) of the ability of flies to respond to a complex stimulus. After 24 hours of SD, all wild-type flies (CS, White 1118 and Oregon R lines) show a reduced ability to respond to a complex stimulus, while the mutant line called “SD resistant 1” is not affected. The ability of flies to respond to the complex stimulus is used as a measure of vigilance.

FIG. 9. Effects of sleep deprivation (SD) of the ability of flies to respond to a thermal stimulus. After 24 hours of SD, all wild-type flies (CS, White 1118 and Oregon R lines) show a reduced ability to respond to a thermal stimulus, i.e. their latency to beam crossing is increased. The latency does not change in the mutant line called “SD resistant 1”. The ability of flies to respond to the thermal stimulus is also used as a measure of vigilance.

FIG. 10. Effects of sleep deprivation (SD) of the ability of flies to respond to a complex stimulus. After 24 hours of SD, all wild-type flies (CS, White 1118 and Oregon R lines) show a reduced ability to respond to a complex stimulus, while the mutant line called “Short Sleeper 1” is not affected. The ability of flies to respond to the complex stimulus is used as a measure of vigilance.

FIG. 11. Effects of sleep deprivation (SD) of the ability of flies to respond to a thermal stimulus. After 24 hours of SD, all wild-type flies (CS, White 1118 and Oregon R lines) show a reduced ability to respond to a thermal stimulus, i.e. their latency to beam crossing is increased. The latency does not change in the mutant line called “Short Sleeper 1”. The ability of flies to respond to the thermal stimulus is also used as a measure of vigilance.

FIG. 12. Effects of sleep deprivation (SD) of the ability of flies to respond to a complex stimulus. After 24 hours of SD, all wild-type flies (CS, White 1118 and Oregon R lines) show a reduced ability to respond to a complex stimulus, while the mutant line 5707, in which one of the two copies of the gene Ork1 is missing, is not affected. The ability of flies to respond to the complex stimulus is used as a measure of vigilance.

FIG. 13. Effects of sleep deprivation (SD) of the ability of flies to respond to a thermal stimulus. After 24 hours of SD, all wild-type flies (CS, White 1118 and Oregon R lines) show a reduced ability to respond to a thermal stimulus, i.e. their latency to beam crossing is increased. The latency does not change in the mutant line 5707. The ability of flies to respond to the thermal stimulus is also used as a measure of vigilance.

FIG. 14. Table of quantitative PCR results illustrating TREK-1 mRNA expression levels as assayed according to Example 3.

### DETAILED DESCRIPTION OF THE INVENTION

General

Advantages of Drosophila as a model system. Drosophila neurobiology is sufficiently complex to permit meaningful generalizations to mammals and humans: for example, it needs sleep just as we do and it needs to perform in several tasks with undiminished vigilance. At the same time, Drosophila genetics is simple enough to permit a rapid mutagenesis screening and the efficient genetic characterization of the relevant mutants.

The majority of fly genes are shared with humans. In fact, it is becoming increasingly apparent that the vertebrate genome arose from the amplification of a core set of genes not much larger than that of the fly. For instance, the sequence diversity between the various potassium channels is greater within either Drosophila or mouse than the divergence of a particular channel between Drosophila and mouse (Miklos and Maleszka, 2000). Many of the genes involved in human diseases have fly counterparts, and the expression of human genes into flies very often results in phenotypes that mimic human diseases (e.g., human α-synuclein in Drosophila causes a phenotype that resembles human Parkinson's disease). In addition to myriad similarities in cellular structure and function, humans and flies share pathways for intercellular and intracellular signaling (from membrane receptors and ion channels to nuclear transcription factors), developmental patterning, learning and behavior, as well as tumor formation and metastasis, to give just a few examples (see e.g. Littleton and Ganetzky, 2000). Thus, flies are now taken as simplified versions of vertebrate animals rather than simply as models of themselves.

Flies lend themselves ideally to forward genetic approaches, i.e. to the identification of new mutants for the phenotype of interest. For instance, genetic screenings in Drosophila have been invaluable for our current understanding of the basic mechanisms of circadian rhythms, learning and memory, stress resistance and longevity, neurodegeneration and of behavioral sensitization to drugs of addiction (see e.g. Waddell and Quinn, 2001). Fly genome contains approximately 13,600 genes, all of which have been sequenced and annotated (Adams et al., 2000; Rubin et al., 2000). It appears that the fly genome contains much less redundancy than mouse or human genomes. As a rule of thumb, where in the fly there may be only one gene for any given category or function, in mice or humans the number of relevant genes is 3-4 times higher. This means that a mutation in the fly is much more likely to yield an interesting phenotype. Drosophila also offers a much more efficient way of obtaining conditional expression of particular genes in space and time, as well as of producing double or triple mutants. Most importantly, unlike genes of humans or mice, each fly gene can be mutated and subjected to detailed functional analysis within the context of an intact organism in a very short period of time (generation time in flies is approximately 2 weeks). Finally, Drosophila husbandry is cheap, efficient and extremely practical, making it possible to simultaneously screen thousands of flies for the phenotype of interest.

Sleep and vigilance in Drosophila. In order to exploit the many advantages of Drosophila as a model system, it is necessary to demonstrate that Drosophila shows the phenotypes of interest. Indeed, a recent series of experiments carried out in the inventors' laboratory (Shaw et al., 2000a; Greenspan et al., 2001) as well as in another laboratory (Hendricks et al., 2000) have shown that sleep in flies shares all the key features of sleep in mammals. As more extensively discussed in the next sections, these experiments have demonstrated that, like mammalian sleep, prolonged rest in Drosophila is characterized by increased arousal threshold (it takes a louder noise to arouse flies when asleep than when awake) and is homeostatically regulated (flies need to sleep more after being deprived of sleep), independently of the circadian clock. In higher order animals sleep is classically defined using electroencephalographic parameters, e.g. recording brain waves from the skull during periods of sleep and waking. In most cases, EEG recordings allow to distinguish a sleeping animal from an awake animal even in the absence of any other information. The inventors have recently succeeded in measuring brain electrical activity in the fruit fly, and have shown that such activity also changes as a function of sleep and waking (Nitz et al., 2002). As in mammals, sleep is abundant in young flies and is reduced in older flies (Shaw et al., 2000a). Most important for the purposes of the present invention, the inventors have shown that Drosophila sleep is modulated in much the same way as in mammals by stimulants such as caffeine and by hypnotics such as antihistamines (Shaw et al., 2000a).

Similarities between mammalian and fly sleep extend to the molecular level. In a series of systematic studies of gene expression changes across behavioral states, the inventors have demonstrated that the expression of several genes is modulated by sleep and waking in the mammalian brain (Cirelli and Tononi, 1998, 2000a,b). The expression of many of the same genes, e.g. the mitochondrial gene cytochrome oxidase C subunit I, the chaperone/heat shock protein BiP, and genes coding for enzymes implicated in the catabolism of monoamines, is also modulated by sleep and waking in the fruit fly (Shaw et al., 2000a).

Experimental Results Demonstrating Sleep in Drosophila. Although the circadian organization of the rest-activity cycle in Drosophila has been well characterized for several years, it was not known until recently whether in flies the sustained periods of rest during the night constitute a sleep-like state or mere inactivity. Thus, it was important to establish that flies sleep, much as other animals and humans do. This was achieved using behavioral, pharmacological, molecular, genetic and, very recently, classical electrophysiological techniques. (Shaw et al., 2000a; Nitz et al., 2002).

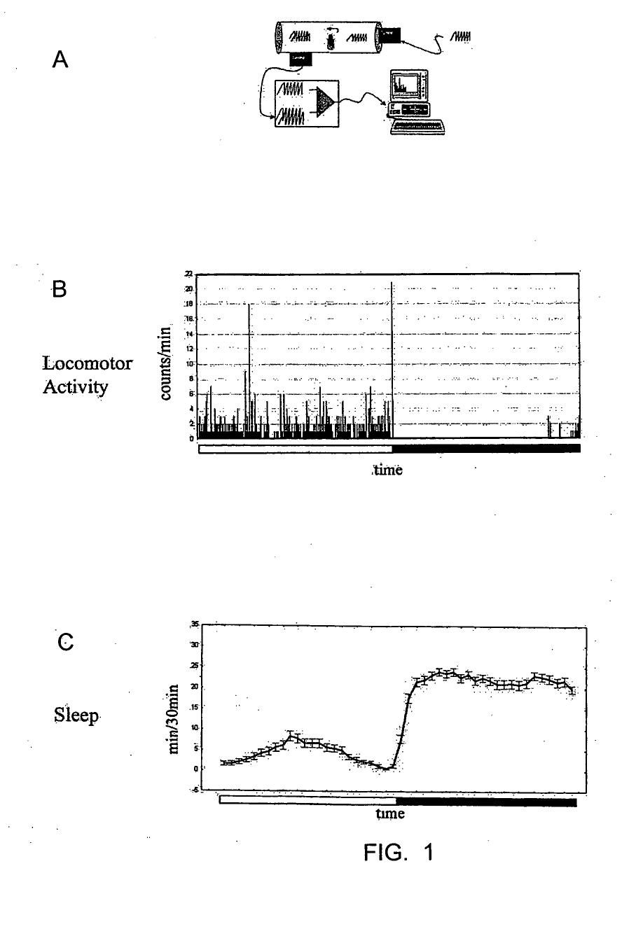

First, an ultrasound activity monitoring system was used to obtain a continuous, high-resolution measurement of fly behavior. Such a system is shown in FIG. 1a. This system detects fine movements of the fly's head, wings, and limbs, in good agreement with visual observation.

Like humans, flies exhibited sustained periods of activity and quiescence, with >90% of quiescence (henceforth referred to as rest) occurring during the dark period. Subsequently, to monitor rest-activity patterns in large numbers of flies, an infrared activity monitoring system was used, which confirmed a robust circadian organization of activity and showed good correspondence with the ultrasound system (FIGS. 1B-C). To determine whether periods of rest were associated with increased arousal thresholds, flies were subjected to vibratory stimuli of increasing intensity [0.05 g (acceleration), n=12; 0.1 g, n=10; and 6.0 g, n=8]. Flies that had been behaviorally awake readily responded to intensities of 0.05 g and 0.1 g (90% of trials). Flies that had been behaviorally quiescent for 5 min or more rarely showed a behavioral response to these stimuli (<20% of trials; P<0.001, c²). However, when the intensity of the stimulus was increased to 6 g, all flies quickly responded regardless of behavioral state (P>0.1, c²). Thus, like sleep in mammals, sustained periods of quiescence in Drosophila are characterized by increased arousal thresholds. Any period of rest longer than 5 min was defined as a sleep period.

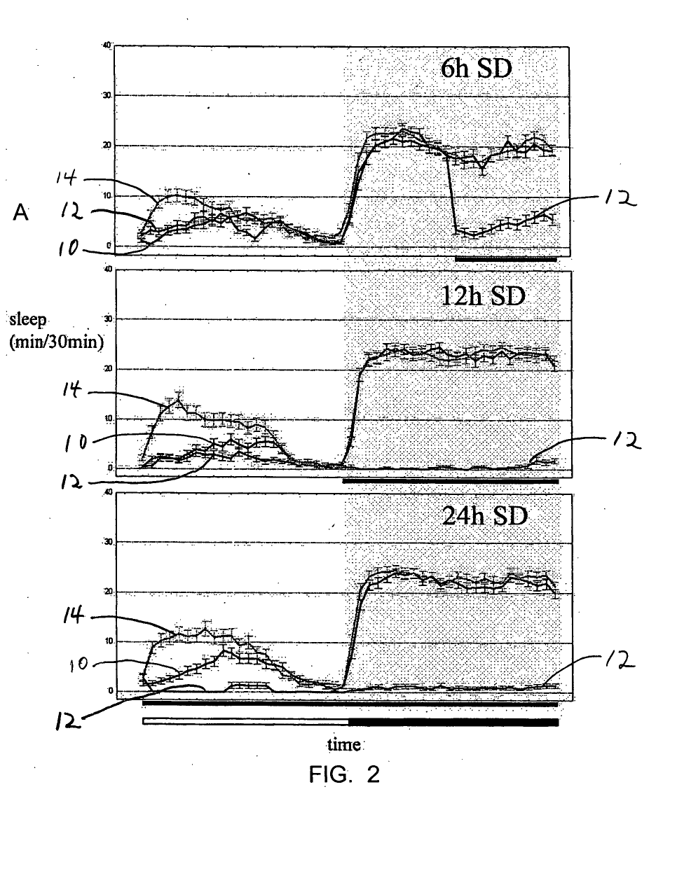

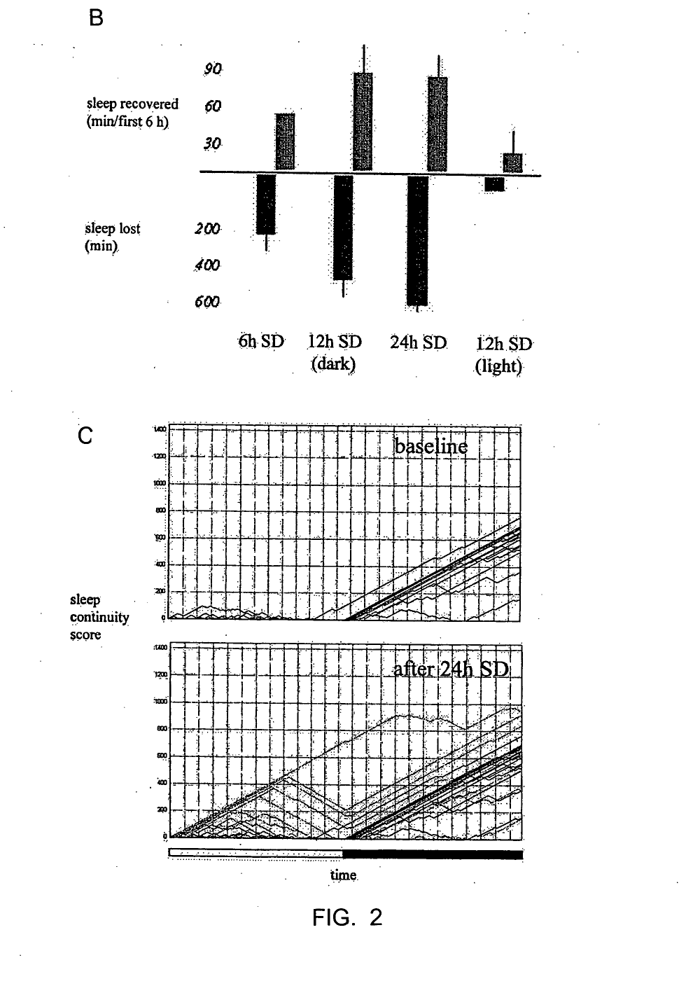

It was further established that the amount of sleep in Drosophila is homeostatically regulated. Flies were deprived of sleep individually by gentle tapping for 12 h. During the first 6 h of the following light period, flies exhibited a significant increase in sleep compared to baseline. In the first 24 h following manual rest deprivation, flies recovered up to 50% of the rest that was lost, a value comparable to the sleep rebound seen in mammals after short-term sleep deprivation. Subsequently, an automated system to sleep-deprive large numbers of flies for 6-24 h was used (FIGS. 2A-C), resulting also in a significant increase in sleep over baseline values during the first 6 h of the following light period. Recordings with the ultrasound system showed that the sleep rebound after deprivation was characterized by actual immobility and not simply an increase of stationary waking activities, such as eating or grooming, which may result in reduced infrared beam crossing. Moreover, the increase in sleep was not accounted for by levels of prior activity. Consistent with this result, when flies were stimulated in the apparatus for 12 h during the light period, sleep not only failed to increase, but was actually reduced by 16±4% during the first 6 h of recovery. Thus, the increase in sleep is not due to physical exhaustion induced by forced activity.

It was also demonstrated that, as in mammals, sleep deprivation in flies affects sleep intensity in addition to sleep duration (Biesiadecki et al., 2003). More precisely, sleep loss in mammals decreases the number of brief awakenings, increases the duration of sleep episodes, and increases arousal thresholds. Similarly in flies, relative to baseline sleep, recovery sleep after sleep deprivation is longer, less fragmented (FIG. 2C), with longer sleep episodes, and associated with increased arousal thresholds.

Next, to investigate whether the homeostatic response is separable from circadian factors, experiments were performed on per⁰¹ mutants, which are arrhythmic under constant darkness because they carry a mutation in one of the major circadian genes, period. In the absence of a circadian rest-activity rhythm, per⁰¹ flies showed a robust homeostatic response following 12 hours of rest deprivation. This indicates that, as in mammals, sleep is homeostatically regulated and can be dissociated from circadian control.

In mammals, sleep is prominent in the very young, stabilizes during adolescence and adulthood, and declines during old age. Sleep in Drosophila follows a similar pattern. On the first full day after eclosion, the amount of sleep is high but declines steadily until day 3, when it reaches an adult pattern. As the flies age, the amount of sleep during the night declines, and by 33 days of age is significantly below that found in young adults. Several studies also indicate that the homeostatic regulation of sleep is preserved in older humans. When 33-day-old flies were deprived of sleep they exhibited a sleep rebound similar to young flies.

Sleep in mammals is modulated by stimulants and hypnotics. For example, caffeine increases waking and motor activity, while antihistamines reduce sleep latency. Flies given caffeine showed a dose-dependent decrease in sleep. By contrast, hydroxyzine, an antagonist of the H1 histamine receptor, increased sleep and reduced its latency (Shaw et al., 2000a; U.S. patent application No. 2002/0042054 A1; and International Publication No. WO 01/38581A2). Thus, two agents that modulate waking and sleep in mammals also modulate vigilance states in Drosophila.

A systematic screening of gene expression in Drosophila was also performed by using mRNA differential display combined with RNase protection assays. RNA was extracted from whole heads of flies that (i) had been spontaneously resting for 3 h during the dark period; (ii) had been rest deprived for 3 h at the same circadian time, or (iii) had been spontaneously awake for 3 h during the light period, thereby making it possible to distinguish between changes associated with behavioral state and those associated with circadian time. Several “waking” genes in the fly corresponded to “waking” genes in the rat (Cirelli and Tononi, 1998, 2000 a,b), including those coding for the mitochondrial gene Cytochrome oxidase C, subunit I and for the endoplasmic reticulum chaperone BiP, which may promote the structural changes necessary for the establishment of long-term memory.

Finally, mRNA levels of arylalkyamine N-acetyltransferase (Dat), an enzyme involved in the catabolism of monoamines, were increased in flies during waking compared to sleep. In rats, waking is associated with a marked increase in brain mRNA for arylsulfotransferase, another enzyme implicated in the catabolism of monoamines. These findings are of importance because waking is associated with high central monoaminergic activity, while a reduction of such activity is a hallmark of sleep. This has led to the suggestion that sleep may serve to counteract the effects of continued monoaminergic discharge. According to this hypothesis, an impaired catabolism of monoamines should result in an increased need for sleep. To evaluate this possibility, a Drosophila mutant was examined, in which the transcriptional level and activity of the Dat enzyme is deficient (Dat^{lo}). Indeed, it was found that the more severely mutant the fly is at the Dat locus, the greater the rebound (Shaw et al., 2000a). Although the mechanisms responsible for the increased homeostatic response to rest deprivation are presently unclear, these results suggest a linkage between the catabolism of monoamines and the regulation of sleep and waking in Drosophila. They also suggest that sleep may subserve the same fundamental function/s in species as different as flies and rats.

Recent experiments in the inventors' laboratory have also shown that local field potentials can be recorded from the brain of Drosophila and that such potentials are modulated by behavioral state (Nitz et al., 2002).

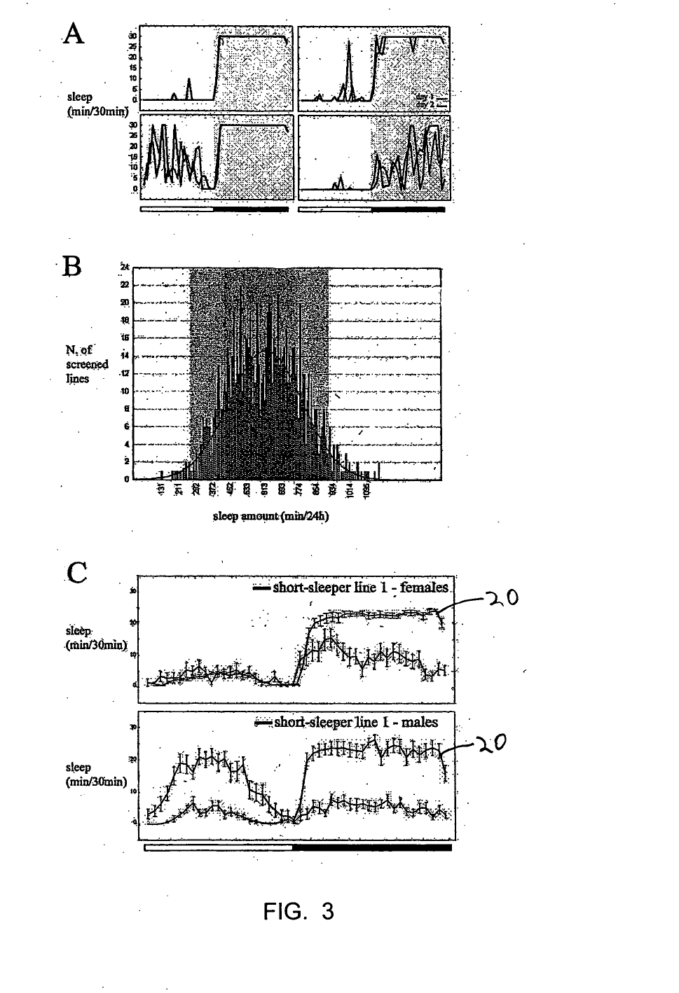

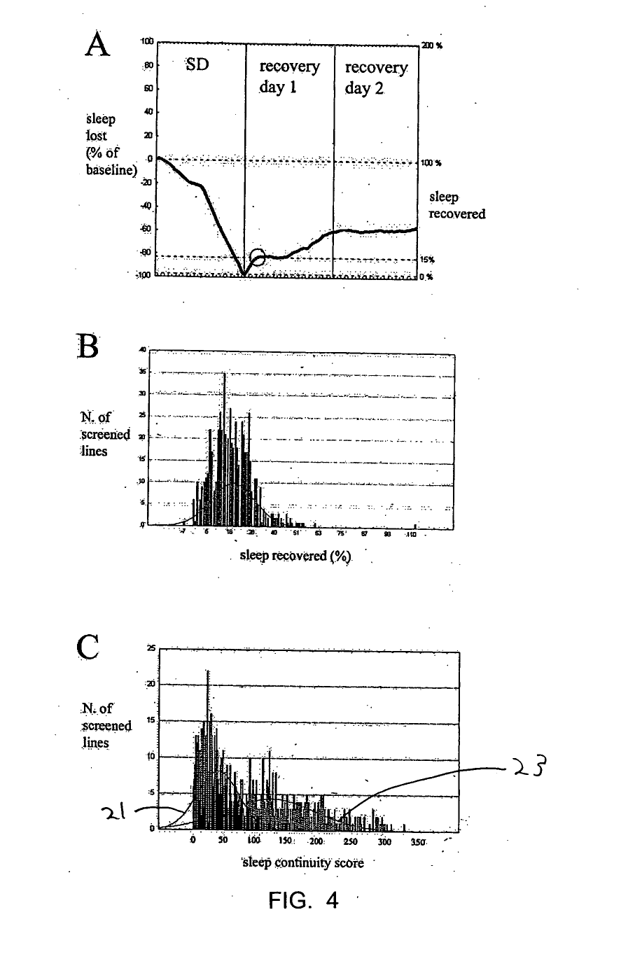

To further evaluate the feasibility of using Drosophila as a model system for the genetic dissection of sleep, sleep patterns and the response to sleep deprivation were examined in several fly lines characterized by mutations of genes expressed in the CNS. (Shaw et al., 2000b; Cirelli et al. 2003; Cirelli 2003). The genes included, but were not limited to, synthetic and catabolic enzymes, protein kinases, receptors, transcription factors, and clock genes. As described below, the inventors also examined sleep patterns in several thousands of EP lines carrying mutations due to random insertion of P elements in the fly genome. Results indicated that the amount of sleep over the 24/h period and the homeostatic response to 24 hours of sleep deprivation were comparable to wild type flies in the vast majority of mutant lines studied so far (Cirelli, 2003). In a few lines, however, the amount of rest over the 24/h period was significantly smaller than in wild-type flies and/or the response to sleep deprivation was significantly abnormal (see FIGS. 3 and 4, discussed in detail below).

To summarize, research conducted in the present inventors' laboratory showed that like mammalian sleep, sleep in Drosophila is characterized by changes in brain electrical activity, increased arousal threshold (it takes a louder noise to arouse flies when asleep than when awake) and is homeostatically regulated (flies need to sleep more after having being sleep deprived). As in mammals, sleep is abundant in young flies and it is reduced in older flies. Sleep in Drosophila is modulated by stimulants (e.g. caffeine) and hypnotics (e.g. antagonist of the H1 histamine receptor). Moreover, several molecular markers modulated by sleep and waking in mammals are also modulated by sleep and activity in Drosophila. The phenotype used to evaluate sleep in a variety of fly mutant lines is stable, thus making a systematic screening for sleep-related mutants in Drosophila possible. Such systematic screening is likely to be successful in identifying lines in which the amount of sleep and/or the response to sleep deprivation is significantly different from wild-type. Molecular targets may then be isolated which will be useful for screening sleep or wakefulness-promoting compounds.

Before the present methods are described, it is understood that this invention is not limited to the particular methodology, protocols, animal subjects, and reagents described, as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a channel” is a reference to one or more channels and equivalents thereof known to those skilled in the art, and so forth.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications and sequence submissions (as referenced by accession nos.) mentioned herein are incorporated herein by reference as if set forth in their entirety for the purpose of describing and disclosing the polypeptides, polynucleotides, vectors, animals, instruments, statistical analysis and methodologies which are reported in the references which might be used in connection with the invention. Nothing herein is to be construed as an admission that the invention is not entitled to antedate such disclosure by virtue of prior invention.

The Invention

I. Screens for Identification of Short Sleep, No Rebound and Sleep-Deprivation Resistant Mutants. In one aspect of the present invention, unique screens for rapidly identifying short sleep, no rebound and sleep-deprivation resistant mutants are provided. In one embodiment, a method for identifying a Drosophila mutant fly with a short sleep phenotype includes the steps of: (a) obtaining a Drosophila mutant; and (b) recording sleep quantity of the mutant over a time course including a baseline period, a sleep deprivation period, and a recovery period, wherein a Drosophila mutant with a short sleep phenotype requires significantly less sleep compared to wild type Drosophila subjected to the same time course.

In another embodiment, a method for identifying a no rebound Drosophila mutant fly includes the steps of: (a) obtaining a Drosophila mutant; and (b) recording sleep quantity of the mutant over a time course including a baseline period, a sleep deprivation period, and a recovery period; and (c) determining the effect of the sleep deprivation period on the sleep quantity during the recovery period in comparison to the sleep quantity for the baseline period wherein a no rebound Drosophila mutant requires significantly less sleep during the recovery period than wild type Drosophila subjected to the same time course.

In yet another embodiment, a method for identifying a sleep deprivation resistant Drosophila mutant includes the steps of: (a) obtaining a Drosophila mutant; and (b) recording sleep quantity and vigilance of the mutant over a time course including a baseline period, a sleep deprivation period, and a recovery period; and (c) determining the effect of the sleep deprivation period on the sleep quantity and vigilance during the recovery period in comparison to the sleep quantity and vigilance for the baseline period wherein a sleep deprivation resistant Drosophila mutant: (i) requires significantly less sleep during the recovery period than wild type Drosophila subjected to the same time course; and (ii) displays vigilance during the recovery period comparable to vigilance during the mutant line's baseline period.

A key advantage of using Drosophila for the purposes of the present invention is that it lends itself exceptionally well to the rapid identification of the relevant phenotypes. While researchers have succeeded in obtaining EEG-like recordings from the fly brain (Nitz et al., 2002), as well as continuous high-resolution measurement of fly activity based on an ultrasound standing wave chamber (Shaw et al., 2000), these methods are impractical for evaluating sleep/waking parameters in a large-scale project. However, the inventors have been able to perfect an automatic high-throughput infrared system, the Drosophila Activity Monitoring System (DAMS) that can measure activity in thousands of flies simultaneously. This method, which the inventors have validated against the ultrasound standing wave chamber (Shaw et al., 2000a), has allowed the inventors to screen a greater number of mutant flies per week. In a preliminary screening of more than 7,000 mutant fly lines, the inventors have also shown that the sleep/waking phenotype is not only well defined, but also stable. Indeed, the inventors have found that in the vast majority of these mutant lines the amount of sleep over the 24 hour period and the homeostatic response to 24 hours of sleep deprivation are comparable to wild type flies (Cirelli, 2003). Thus, the available evidence strongly suggests that Drosophila sleep/waking phenotype is well characterized, stable, and easily and rapidly assessable.

Also, vigilance tests that can assess whether fruit flies can respond in a sustained way to visual stimuli having adaptive relevance have been implemented by the inventors. Adult fruit flies startle and jump when exposed to a complex (visual, acoustic and vibratory) stimulus. Such stimulus may be generated by various methods including, but not limited to, the use of a flap which is vigorously and periodically pushed against containers housing the flies. In a preferred approach, the flap contacts each container for approximately 10 seconds and said contact is delivered randomly every hour at opposing sides of the container. Wild type flies, and the majority of mutant flies tested by the inventors, respond to the stimulus by moving away from the side where the stimulus is delivered. By doing so, they cross the infrared beam and the latency to cross the beam is measured by the monitor. The difference between the mean latency to crossing the infrared beam the minute before (pre-stimulus bin) and the minute after the stimulus (post-stimulus bin) is calculated. Such difference is taken as an indicator of vigilance.

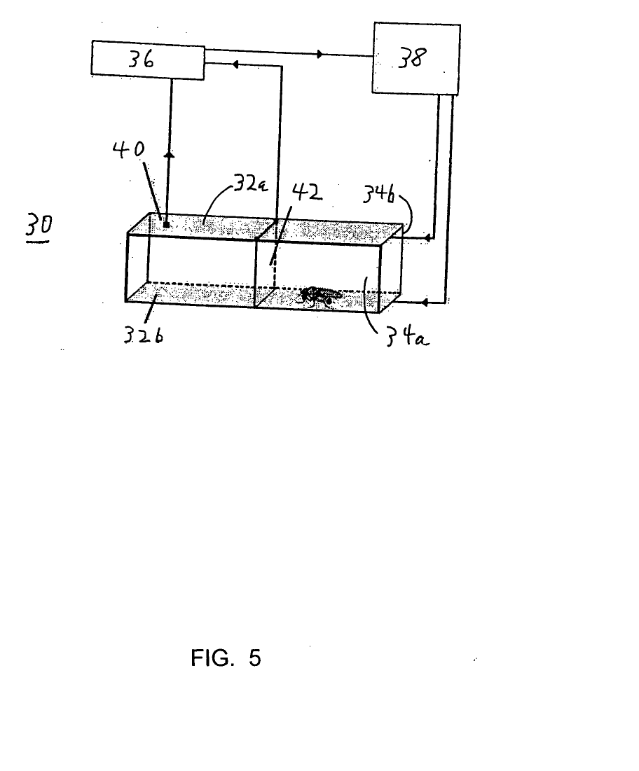

Vigilance and the ability of fly lines to learn and retain memory may also be assayed by using a heat box system, preferably a system as described by Putz et al. (2002). In each heating chamber of this apparatus (FIG. 5), a fly can be conditioned to avoid one side of the chamber if the chamber is heated whenever the fly enters that side. In a subsequent memory test without the application of heat, the fly will demonstrate a retained memory by avoiding the heat-associated chamber side. This system has demonstrated several advantages for assaying memory and learning including: (1) speed and robustness; (2) minimal operator handling requirements for flies; (3) large numbers of flies may be assayed in one experiment; (4) flies are not restrained and therefor damage thereto is minimal; and (5) statistically significant learning curves are generated for individual flies.

Suitable flies for use in the present invention include those available from, for example, the mutation libraries available from the Public Stock Center in Bloomington, Indiana. These lines include deficiency lines as well as lines obtained through insertional mutagenesis with transposable elements (P- and EP elements). In addition, ex-novo chemical mutagenesis with suitable mutagens known in the field, such as ethylmethane sulfonate (EMS), are also useful to generate a multitude of suitable mutants for use in the invention.

The collection of deficiencies lines from Bloomington includes approximately 150 lines, each carrying a deletion of a relatively large portion of the fly genome. The advantage of this collection is that as a whole it covers approximately 80% of the fly genome, thus allowing a comprehensive screening of most of the fly genome. However, each deletion includes many different genes. Should one deficiency line show a sleep exempt or sleep deprivation resistant phenotype, it will be necessary to clarify, through complementation and other genetic means, which gene is responsible for the phenotype. Libraries carrying single gene mutations include the Berkeley Drosophila Genome Project (BDGP) primary collection of 1,045 lines obtained through insertional mutagenesis with P-elements (Spradling et al., 1999), as well as the Rorth collection of 2,300 lines obtained through EP-induced misexpression (Rorth et al., 1998). The first collection contains loss-of-function mutations due to the insertion of a P-element inside a gene, with subsequent destruction of its function. The second collection largely contains gain-of-function mutations due to the insertion of a particular P-element (called EP element) within the 5′ end of a gene, with subsequent overexpression of that gene. An advantage of insertional mutagenesis relative to the use of deficiencies lines is that it allows rapid molecular cloning of the mutated gene, thus greatly facilitating the molecular characterization of the gene of interest once a particular mutant line has been identified. A limitation of this approach, however, is that P-elements do not insert at random into the genome, but have preferred hot spots (Liao et al., 20000). Therefore, significant portions of the genome are likely to be spared by mutations when using insertional mutagenesis. For this reason, in addition to screening P-elements related lines, mutants suitable for use in the invention may also be made using chemical mutagenesis, most preferably EMS, which is known to randomly induce small (point) mutation over the entire genome at a reasonable rate. EMS has been the most frequently used chemical mutagen in Drosophila over the last 25 years, for instance in the search for learning mutants, circadian mutants, and paralytic mutants (Roberts, 1998).

The analysis of both new EMS-produced mutant lines and already available mutant lines (deficiencies, P- and EP element related mutant lines) allows the screening of a large portion of the fly genome. More precisely, using a screen according to the invention, the inventors' laboratory has screened ˜100 mutant lines (16 flies/line) per week, i.e. >5,000 lines on a yearly basis.

Because the entire fly genome has now been sequenced and annotated, it is known in the art to clone a gene from the mutant line of interest. Once the gene has been cloned, it is straightforward to exploit the huge resources of Drosophila's genetics, for example, to over-express the gene in different amounts and/or different regions of the brain, to look for interaction with other genes (e.g. create double mutants), to rescue a loss-of-function phenotype by reintroducing the gene into the genome, etc. Gene products identified subsequent to phenotype screens according to the invention may serve as targets against which sleep or wakefulness-promoting compounds are screened, as described in a later section and claimed herein.

The following provides a more detailed description of the general screening methods described above and is meant for illustrative purposes only and is in no way meant to limit the invention.

A. Choice of mutant lines and EMS mutagenesis screening. The strategy in this particular screening example is to test as many mutant lines as possible, including those already available from the Public Stock Center (Bloomington, Ind.) and from the laboratory of Dr. Barry Ganetzky (Department of Genetics, University of Wisconsin), as well as new mutant lines created by EMS mutagenesis. The already available lines are ˜4,000: ˜150 deficiencies lines, the primary BDGP collection of loss-of function mutations obtained through insertional mutagenesis with P-elements (1,045), the Rorth collection of EP-lines (2,300), and ˜500 mutant lines generated in a previous EMS screening performed in the Ganetzky laboratory (Stem and Ganetzky, 1992).

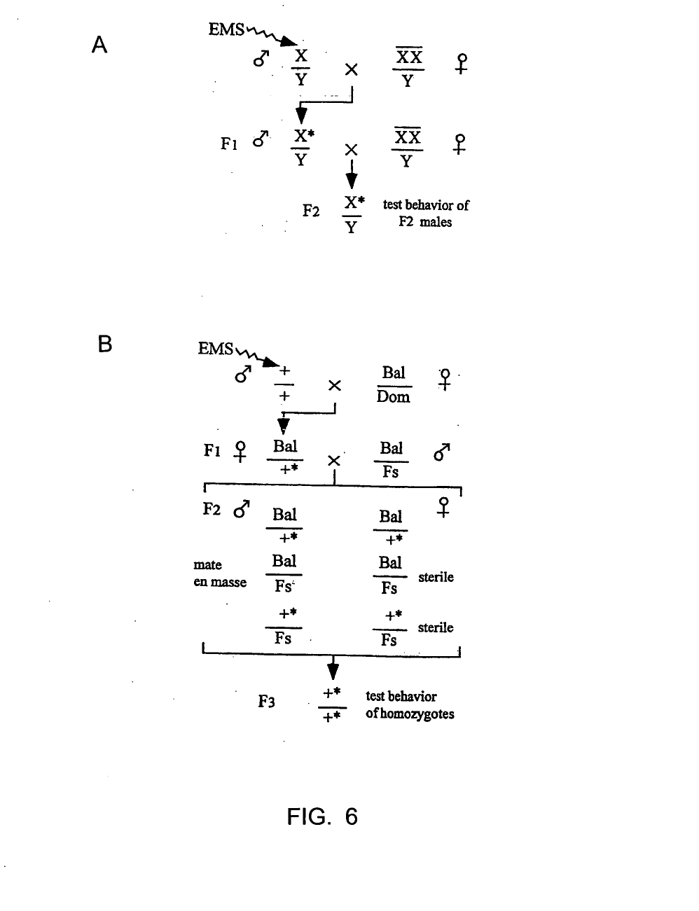

Flies are cultured under standard conditions, preferably at 21° C., 68% humidity, 12 hr:12 hr light:dark cycle, on yeast, dark corn syrup and agar food. Mutant flies are also generated using the EMS mutagenesis screening for X-linked mutations and recessive autosomal mutations. A mating scheme for isolation of recessive X-linked mutations is shown in FIG. 6A. Parental males are mutagenized with ethyl methanesulfonate (EMS) and mated en masse to attached-X females. F1 males are mated individually to attached-X females to establish a line; each F1 male represents an independently mutagenized X chromosome. Thus, to test 1000 mutagenized X chromosomes, 1000 separate F1 crosses would be set up. From each such cross the resulting F2 sons are genetically identical to each other and carry the same mutagenized X.

A mating scheme for isolation of recessive autosomal mutations is shown in FIG. 6B. The same scheme can be used for either of the major autosomes. Bal represents a dominantly marked, multiply inverted, recessive lethal balancer chromosome. Dom represents a dominantly marked chromosome. Fs is a dominant female-sterile mutation. Females carrying this mutation cannot reproduce; males are unaffected. Single females are used to start each F1 line. Each such line represents a different mutagenized chromosome. To test 1000 mutagenized autosomes, 1000 separate F1 crosses would be set up. The resulting F2 progeny from each separate line are transferred en masse and allowed to mate at random. Because of Fs only the desired Bal/+* females are fertile. Random mating thus results in the production of a balanced stock and the segregation of homozygous +*/+* progeny in the F3 generation that can be tested for the desired behavior.

B. Time courses for phenotypic screening and assays for vigilance. As previously discussed, the invention relies on motor activity to monitor the fly sleep/waking pattern. In one particular particularly preferred embodiment of the invention, motor activity is measured using the Drosophila Activity Monitoring System (DAMS). The DAMS is generally described in International Published Patent Application No. WO 01/38581; U.S. patent application No. 2002/0042054 A1. The system includes a Macintosh computer, a power supply unit, and one or more activity monitors (e.g., 120 individual monitors). In one embodiment, each monitor contains counting circuits and snap-in clips to support 32 6.5-mm glass tubes (5 mm I.D.), each containing one fly. As each fly moves back and forth in its tube, it interrupts an infrared light beam that bisects the tube. This interruption is detected by the microprocessor in the monitor and is registered as a “count” for that particular tube (channel). The monitor watches the beams for all 32 channels simultaneously, recording counts accordingly as they occur. Every minute the computer commands all monitors simultaneously to “freeze and hold” their current count totals and to begin counting from zero for the next count period. The computer then interrogates each monitor in turn, requesting that the accumulated “frozen” counts for each channel be transmitted over the data bus. The computer stores the data in a file on its hard disk for subsequent transfer to archival storage and post-collection analysis.

In identifying sleep deprivation resistant mutants, cognitive performance (vigilance) is measured using at least one of the vigilance assays previously described. As discussed before, these tests need to be repeated several time to assess the effects of sleep deprivation on sustained cognitive performance. For example, the assay using a complex stimulus is performed while keeping the flies inside the glass tubes. Flies remain inside a DAMS monitor. The stimulus is produced by a flap vigorously pushed against the glass tubes housing the flies, and is delivered via a computer every hour at either side of the tubes. The mean latency (in sec) to crossing the infrared beam after the stimulus was delivered is measured and compared with the latency when the stimulus was not present. Flies are tested for a total of 48 hours, including one baseline day and the first recovery day after SD.

In a second vigilance test, vigilance is measured after flies are exposed to a thermal stimulus. For this test, single flies are removed from the glass tubes and placed inside a heat box to assess spatial memory in Drosophila (Putz et al., 2002). The heat box includes 16 heating chambers, each housing a single fly (FIG. 5). Inside each chamber, position and movements of the fly are recorded and displayed on line. Flies are first adapted to the chamber for at least 30 min. Temperature on either side of the chamber is then alternately increased by 4° C. every min from 22° C. (baseline value) to 44° C. The latency to crossing the infrared beam, i.e. the time a fly needs to move to the cooler side of the chamber, is measured for each temperature step. Latencies for all temperature steps are averaged for each fly. Flies are tested during the first 2 hours after the end of SD and at the same circadian time during baseline.

C. Sleep deprivation. Flies are sleep deprived in the DAMS monitors without removing them from the glass tubes. At the start of the SD period monitors are placed vertically within a framed box able to rotate along its major axis under the control of a motor. The box can rotate 180° C. clock-wise or counter-clock-wise (2-3 revolutions/min). At the nadir of each rotation, the monitors are dropped 1 cm. This causes the flies to fall from their current position to the bottom of the tube. This method has been proven to be effective in reducing total sleep time by 90% during the 24 hour sleep deprivation period.

The motor activity of each fly mutant line (n=16 flies/line) may be continuously recorded in the DAMS monitors for one week, including 3 baseline days (B1-3), 1 or more sleep deprivation night (SD), and 1-3 days of recovery after sleep deprivation (R1-R3).

D. Data analysis. Data analysis according to the invention includes measures of the quantity of sleep (S) and waking (W) and of their distribution over the 24-h period, as well as measures of S and W quality. As described above, DAMS measures activity as counts (number of crossings) per minute and S and W are determined for consecutive 1 min epochs. W is defined as any period of at least 1 minute characterized by activity (one or more counts per minute). S is defined as any period of uninterrupted behavioral quiescence (no counts/min) lasting for at least 5 min. Mean values of the amount of S are calculated on consecutive 30-min time intervals and the time course of the amount of S is graphically shown over the entire day (e.g., FIG. 1C). In mammals, slow wave activity (SWA), i.e. the EEG power density in the 0.75-4 Hz range, is widely considered to be a reliable marker of sleep intensity.

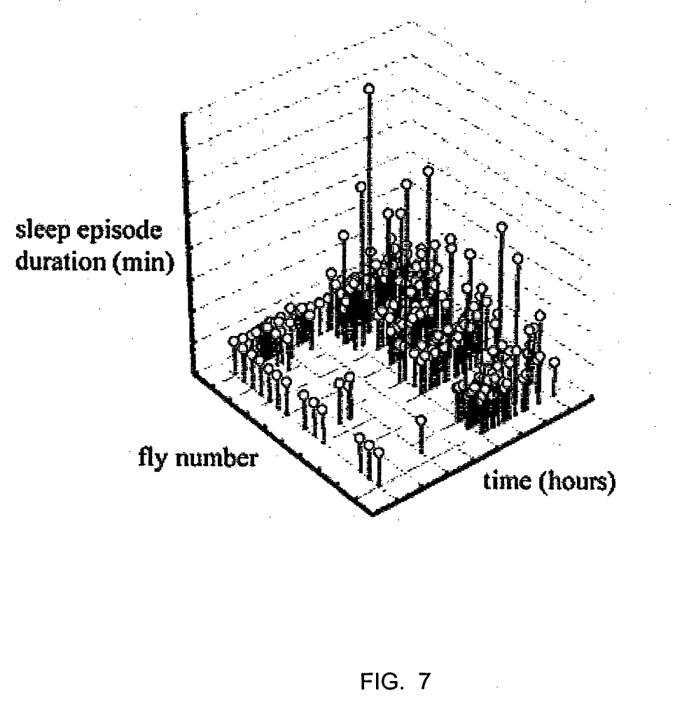

Another reliable marker, which can be readily measured in flies, is sleep fragmentation. In several mammals the increase in SWA during recovery after sleep deprivation is associated and negatively correlated with a decrease in the number of brief awakenings (a measure of sleep fragmentation; refs in Huber et al., 2000). Sleep fragmentation in flies is measured using three parameters, the number of brief awakenings, the duration of sleep episodes, and the sleep continuity score (S-score). Brief awakenings are defined as 1-min epochs of wakefulness. The duration of sleep episodes is calculated by counting the number of consecutive 1 min epochs of sleep (e.g., FIG. 7). The sleep continuity score for a given time interval (e.g. 3 h and 24 h) is calculated by assigning a value of 1 to each 1 min epoch of sleep, and a value of −1 to each 1 min epoch of waking. Negative values are automatically reset to 0. The sleep continuity score is high if sleep is continuous and undisturbed, and low if sleep is fragmented (FIG. 2C). Finally, the amount of behavioral activity (counts/min) over the 24-h period is also measured, as a measure of W. The effect of SD on each of the parameters discussed above is assessed by comparing the values from R1-R2 with those from B2-B3. To emphasize differences in the effects of SD between different mutant lines and wild-type flies, the recovery-baseline differences from different lines are presented visually on the same graph.

Short sleep, no rebound and sleep deprivation resistant phenotypes are preferably identified according to the following criteria. If the quantity of sleep during a time course including a baseline period is significantly less than required by wild type Drosophila lines subjected to the same time course, then the mutant line is identified as having a short sleep phenotype. Examples of short sleep phenotype mutants are shown in FIG. 3. More preferably, the criteria further call for the baseline period for a mutant line to be on average less than approximately 375 minutes/day, replicated over at least three experiments, compared to approximately 700 minutes/day in wild type Drosophila for the baseline period.

If a Drosophila mutant line subjected to a time course including a baseline period, sleep deprivation period and recovery period requires significantly less sleep during the recovery period than wild type Drosophila subjected to the same time course, then the mutant line possesses a no rebound phenotype. Examples of such mutant lines are shown in FIG. 4. More preferably, in the case where the baseline period, sleep deprivation period and recovery period are each 24 hours in length, if the quantity of rebound sleep for a mutant line during the 24 hour recovery period immediately after sleep deprivation is on average less than approximately 3.0% higher than during the baseline period, replicated over at least three experiments, compared to wild type Drosophila showing, on average, an increase of approximately 25%, then the mutant line is identified as possessing a no rebound phenotype.

If a Drosophila mutant line subjected to a time course including a baseline period, sleep deprivation period and recovery period: (i) requires significantly less sleep during the recovery period than wild type Drosophila subjected to the same time course; and (ii) displays vigilance during the recovery period comparable to vigilance during its baseline period, then the mutant line possesses a sleep deprivation resistant phenotype. More preferably, in the case where the baseline period, sleep deprivation period and recovery period are each 24 hours in length, if the quantity of rebound sleep for a mutant line during the 24 hour recovery period immediately after sleep deprivation is on average less than approximately 3.0% higher than during the baseline period, replicated over at least three experiments, compared to wild type Drosophila showing, on average, an increase of approximately 25%, and the mutant line shows a performance on vigilance tasks statistically similar to that observed during the baseline period for the respective mutant line, then the mutant line is identified as possessing a sleep deprivation resistant phenotype. Vigilance between individual flies varies and therefore comparisons related to vigilance must be based on an individual fly's vigilance performance during baseline and recovery periods. Thus, a sleep deprivation mutant not only requires less sleep during the recovery period as compared to wild type flies but also maintains cognitive performance abilities during the recovery period substantially unreduced to those before sleep deprivation, for the conditions defined above.

Depending on the mutant line, it may or may not be necessary to clone the gene of interest. Such cloning and characterization are carried out by techniques widely known in the art. Once the gene has been identified, appropriate genetic analysis, including complementation tests may be carried out, to confirm that the identified locus is responsible for the phenotype of interest. The molecular characterization may include sequence analysis of the gene and the construction of transgenic lines in which the gene of interest is expressed constitutively throughout development or is induced at selective times by heat shock. Moreover, cDNA probes may be synthesized to conduct expression analysis with northern blot and in situ hybridization experiments, and specific antibodies may be produced against the corresponding protein product.

II. Methods for identification of sleep or wakefulness-promoting compounds. The present invention further encompasses methods for isolating candidate sleep or wakefulness-promoting compounds. In one embodiment of the invention, a method for identifying a compound useful for increasing wakefulness in an organism requiring sleep includes the step of determining the effect of a test compound on a two pore domain K+ channel to thereby identify a compound which decreases the K+ current or shortens the open state of said two pore domain K+ channel wherein the decreased K+ or shortened current correlates with increased wakefulness in said organism.

In another embodiment, a method for identifying a compound useful for promoting highly restorative sleep in an organism requiring sleep is provided including the step of determining the effect of a test compound on a two pore domain K+ channel to thereby identify a compound which increases the K+ current or prolongs the open state of said two pore domain K+ channel wherein the increased K+ current or prolonged open state correlates with promoting highly restorative sleep in said organism.

Available pharmacological approaches to prolong wakefulness while preserving sustained cognitive performance have largely failed. Available wakefulness-promoting drugs have side effects and are not effective after sustained periods of sleep deprivation. Similarly, none of the hypnotics currently available is able to mimic all the physiological aspects of sleep and to concentrate them in a short and yet highly restorative sleep.

It would be extremely desirable to obtain a new class of pharmacological agents capable of prolonging wakefulness or inducing and maintaining sleep in a highly efficacious and specific manner. Recent studies have conclusively established that, during slow wave sleep, the membrane potential of all cells in the cerebral cortex undergoes slow oscillations, which includes a hyperpolarization phase characterized by the interruption of neuronal firing. Under natural conditions, the transition between waking and sleep and the emergence of slow oscillations is governed by the reduced firing of brain neuromodulatory systems with diffuse projections, such as the noradrenergic system, the cholinergic system, the histaminergic system and several others. In the cerebral cortex and the thalamus, the reduced firing of neuromodulatory systems produces changes in several ion currents (conductances) that influence cell excitability and membrane potential.

One such conductance is a leak or background potassium (K+) conductance. When this conductance is increased, cells, become hyperpolarized, and several other changes follow that lead to slow oscillations and other rhythmic activities of sleep. Until recently, there was an absence of molecular counterparts for leak channels. However, the situation has changed after the cloning of the K+ channels TOK1 from Saccharomyces cerevisiae and KCNKO from Drosophila melanogaster. KCNK0 is now understood to be a prototype of a new category of K+ channels that possess two pore-forming domains in each subunit (two pore domain K+ channels). Since then, over fifty genes for two pore domain subunits have been recognized in sequence databases and more than fourteen of them have been cloned and intensively studied.

Example 2 set forth below describes the present inventors' discovery that KCNK0 (also known as ORK1) plays an important role in sleep and wakefulness. Mutant flies harboring deficiencies spanning the region in which the ORK1 gene is located displayed only 375 minutes of sleep per day as compared to wild type flies averaging approximately 700 minutes of sleep per day. Lack of the gene encoding ORK1 appears responsible for a unique short sleep phenotype and suggests the importance of two pore domain K+ channels in sleep regulation. The amino acid sequence for ORK1 is set forth in SEQ ID NO: 1. The ORK1 amino acid sequence is available at accession no. NM167259.

Another important aspect of two pore domain K+ channels is that they remain on or off for long periods of time (several minutes or more). Yet another important aspect is that several two pore domain K+ channels are highly expressed in the brain, including brain regions that need sleep. In at least some cases, the opening of some two pore domain K+ channels is known to be controlled by neuromodulators such as those influencing sleep and wakefulness. Finally, some two pore domain K+ channels are affected by general anesthetics that often produce neural oscillatory activity similar to that observed in slow wave sleep.

Based on the above, hyperpolarization observed during the slow oscillation characteristic of slow wave sleep is likely due to an increased K+ conductance through two pore domain K+ channels expressed in brain regions such as the cerebral cortex and the thalamus. Besides the previous considerations, this is supported by the observation that, during slow wave sleep, membrane conductance is on average higher than in waking, being almost twice as large during the hyperpolarization phase. Moreover, the fact that leak currents mediated by two pore domain K+ channels remain on or off for long periods of time is precisely what is needed to control long-lasting changes in behavioral states such as sleep vs wakefulness.

It is likely that an increased value of K+ leak currents through two pore domain K+ channels in the brain is an essential feature of slow wave sleep. An increased conductance of two pore domain K+ channels would be responsible for triggering slow oscillations, delta waves, sleep spindles and other phenomena that are characteristic of slow wave sleep.

Therefore, the modulation of K+ leak conductances through two pore domain K+ channels in cortical and thalamic neurons is likely the final common pathway mediating the action of most factors promoting wakefulness or sleep. Such factors include neuromodulatory systems with diffuse projections mediating arousal, circadian changes in sleep propensity, and the increased sleep pressure that occurs after prolonged wakefulness.

Therefore, an effective and specific way of prolonging wakefulness or inducing and maintaining sleep, respectively, is to develop specific pharmacological agents capable of opening or closing two pore domain K+ channels that: i) are expressed in brain regions such as cerebral cortex and thalamus; ii) are sensitive to modulation by neuromodulatory systems acting through second messengers and possibly a phosphorylation step; and iii) are sensitive to general anesthetics. Specifically, based on the criteria listed and on experimental results presented in Examples 2 and 3, the inventors have hypothesized that it is likely that the two pore domain K+ channel known as TREK-1 is a key mediator of sleep (when open) and waking (when closed). Consistent with the hypothesis, TREK-1 is highly expressed in much of the rat brain; it is inhibited by protein kinase A and protein kinase C-mediated phosphorylation; it is activated by volatile general anesthetics, including isoflurane, halothane, diethylether, and chloroform; it is expressed at higher levels in the brain of sleeping animals (as shown and described in Example 3). Thus, TREK-1 is an extremely desirable molecular target against which test compounds may be screened to determine their abilities to modulate K+ currents and, consequently, promotion of sleep or wakefulness. As used herein, the term “TREK-1” is meant to encompass known homologs including, but not limited to, Mus musculus TREK-1 K+ channel subunit (Accession No. U73488) and Homo sapiens TREK-1 K+ channel subunit (KCNK2; Accession No. NM014217). The amino acid sequence for Homo sapiens TREK-1 is set forth in SEQ ID NO:2.

In general, homologs of TREK-1 are certainly envisioned to be useful in the methods described and claimed herein. In addition to TREK homologs, other two pore domain K+ channels are useful in the present invention and include proteins such as Drosophila ORK1 (SEQ ID NO: 1). A relationship between sleep/wakefulness and the ORK1 two pore domain K+ channel has been observed by the present inventors and is described in Example 1. ORK1, as well as ORK1 homologs, are additionally-contemplated as being useful two pore domain K+ channels for carrying out the present invention.

Specific agonists or antagonists of two pore domain K+channels, such as TREK-1, are not available. Apparently, no such agents have been considered as potential wakefulness-promoting or sleep promoting factors. Compounds suitable for screening against two pore domain K+ channels according to the present invention may derive from a wide variety of sources including compound libraries commercially available. Identification of lead compounds may be followed by rational drug design according to techniques well known in the art for deriving more potent and/or more stable derivatives.

Potassium channel screening assays are generally carried out in vitro utilizing techniques known in the art for monitoring and recording K+ currents (e.g., electrophysiology techniques including patch clamping). In a preferred method, a ⁸⁶Rb screening assay using host cells such as CHO cells stably expressing selected potassium channels is utilized with currents being monitored by standard patch clamping techniques. A test compound is introduced to the cells and effects on K+ currents, if any, are monitored by patch clamp. As a differential screening approach, a fibroblast L-cell line expressing, for example, the cloned human Kv1.5 potassium channel may be used to select compounds that have more selectivity for the neuronal type potassium channels versus the cardiac potassium channels. A high throughput in vitro screen amendable to use with the present invention is described in International Publication WO 99/66329, incorporated herein by reference.

III. In vivo screening and administration of sleep-related compounds. The present invention further includes methods for in vivo use of candidate compounds by the administration of the respective compounds to organisms requiring sleep. Such administration allows for assaying the effects of the compounds on sleep and/or wakefulness. Thus, one embodiment of the invention is a method for increasing wakefulness in an organism requiring sleep including the step of administering an effective amount of a wakefulness-promoting compound identified to the organism and thereby increasing the wakefulness in said organism. In another embodiment, a method is provided for promoting highly restorative sleep in an organism requiring sleep including the step of administering an effective amount of a sleep-promoting compound to the organism and thereby promoting highly restorative sleep in said organism.

The invention is also directed to in vivo methods for identifying a wakefulness-promoting compound, comprising the steps of: (a) administering a test compound to wild type (wt) Drosophila; and (b) recording sleep quantity and vigilance of said wt Drosophila over a time course including a baseline period, a sleep deprivation period, and a recovery period whereby a wakefulness-promoting compound is identified as bestowing upon said wt Drosophila wakefulness wherein said wt Drosophila administered the test compound: (i) requires significantly less sleep during the recovery period than wt Drosophila not receiving the test compound and subjected to the same time course; and (ii) displays vigilance during the time course comparable to vigilance displayed by awake wt Drosophila not receiving the test compound.

The present invention further encompasses in vivo methods for identifying a continuous performance (CP) compound, comprising the steps of: (a) administering a test compound to wild type (wt) Drosophila; and (b) recording sleep quantity and vigilance of said wt Drosophila over a time course including a baseline period, a sleep deprivation period, and a recovery period; wherein a CP compound is identified as bestowing upon said wt Drosophila sleep deprivation resistance wherein said wt Drosophila: (i) requires significantly less sleep during the recovery period than wild type Drosophila not receiving the test compound and subjected to the same time course; and (ii) displays vigilance during the recovery period comparable to vigilance during the baseline period.

In vivo testing may be performed in sleep requiring organisms including flies and rats, depending on the characteristics of the compound. For in vivo testing of compounds in flies, drugs may be mixed with the food and sleep/waking phenotype and vigilance studied with the DAMS system as previously described, except that wild type (Canton-S) flies may be used. Testing in flies offers the advantage of rapidly evaluating large numbers of individuals (16 or more per compound) in a practical and inexpensive manner.

Candidate compounds, once identified as displaying sleep-related activities by the present methods, may then be tested by lengthier and costlier procedures in normal rats. The rat has been chosen as the experimental animal because the physiological changes during the 24 h sleep-waking cycle and the response to forced waking induced by sleep deprivation have been extensively studied in this species, both in inbred (e.g. WKY) and outbred (e.g. Sprague Dawley) strains. In addition to sleep/waking parameters similar to those used for the flies, rats will be continuously recorded using implanted EEG and EMG electrodes (24 hours/day for several days) to evaluate effects of the candidate compounds on EEG features of sleep and wakefulness, including spectral characteristics. For in vivo testing of candidate compounds in rats, drugs will be administered either systemically or after i.c.v. injections. Rats will be implanted for chronic EEG and EMG recording and recorded in soundproof recording cages for 1-3 weeks. A battery of behavioral tasks will be used to test the efficacy of candidate compounds in counteracting the detrimental effects of sleep deprivation including effects on cognitive performance. Several tasks have been developed in animals to assess sustained attention, i.e. the ability to respond to the occurrence of rare and unpredictable events over prolonged periods of time (e.g. Bushnell, 1998). As discussed previously, these tasks are among those mostly affected by sleep loss. Detection and discrimination paradigms that have been extensively validated in the past to assess sustained attention (Sarter and McGaughy, 1998) may be utilized. A continuous vigilance-sustained attention test may also be employed. Studies in rats are ideal to evaluate in detail polysomnographic features of the candidate compounds, dose-response curves, and toxicity. Moreover, testing in rats opens the way to employing a large battery of protocols used to assess cognitive performance. Testing in rats also represents a necessary step towards evaluating the candidate compounds in primates and humans.

Advantages and unique features of the present invention include, but are not limited to the following.

Large-scale mutagenesis offers a radically new way of identifying molecular targets for developing sleep and wakefulness-promoting compounds based upon the recent discovery that sleep occurs in Drosophila.

Performing large-scale mutagenesis in Drosophila is the fastest, most practical and cost-effective way of identifying short sleep (sleep exempt) and sleep deprivation resistant mutants and thereby new targets for drug development.

The fly genome has much less redundancy than the mouse genome, thereby providing a much higher likelihood of identifying relevant phenotypes.

The likelihood of obtaining a desirable molecular target is expected to be high based on the success of previous work with fly mutagenesis to elucidate targets involved in circadian rhythms, stress, aging, learning and memory, and other basic biological functions.

Each of the ˜14,000 fly genes has now been sequenced and annotated. Therefore, once a mutant line of interest is identified, subsequent molecular and genetic manipulation of the gene of interest is straightforward.

Flies offer an unparalleled flexibility for genetic manipulations, including the production of double and triple mutants, conditional mutants, expression specific mutants, etc.

Thousands of mutant lines, each affecting the expression of one specific gene, are either already available or can be generated and tested within a brief time period. Since generation time in flies is approximately 2 weeks, less than 2 months are required to generate mutant flies ready to be screened. Hundreds of mutant lines can be generated each week and tested 5-7 weeks later.

The first phenotype of interest, fly sleep, is stable and shares all key the features of the human sleep phenotype.

The second phenotype of interest, performance in tasks requiring vigilance, can be tested in flies with sensitive assays such as, but not limited to, the vigilance assays described herein.

Initial in vivo testing of candidate compounds can be performed both in flies, where it is fast and practical, and in rats, where it can be refined to examine a large number of neurobiological effects.

The sleep or wakefulness-promoting compounds isolated according to the present invention are expected to act on radically new targets implicated in the fundamental mechanisms of sleep, waking, and sustained performance in vigilance tasks rather than constituting refinements of already available compounds.

The inventors have determined that an effective and specific way of prolonging wakefulness or inducing and maintaining sleep, respectively, is to develop specific pharmacological agents capable of opening or closing two pore domain K+ channels. Preferably, these channels: i) are expressed in brain regions such as cerebral cortex and thalamus; ii) are sensitive to modulation by neuromodulatory systems acting through second messengers and possibly a phosphorylation step; and iii) are sensitive to general anesthetics.

The inventors have further identified that it is likely that the two pore domain K+ channel known as TREK-1 is a key mediator of sleep (when open) and waking (when closed). TREK-1 is highly expressed in much of the rat brain; it is inhibited by protein kinase A and protein kinase C-mediated phosphorylation; it is activated by volatile general anesthetics, including isoflurane, halothane, diethylether, and chloroform; it is expressed at higher levels in the brain of sleeping animals (see Example 3).

The following examples are illustrative and in no manner limiting of the present invention, the scope of which is set forth in the appended claims.

### EXAMPLES

### Example 1

### Identification of Sleep-Related Mutants

Sleep in the fruit fly is: 1) consolidated into long period of quiescence, 2) characterized by increased arousal threshold, 3) homeostatically regulated; 4) modulated by stimulants and hypnotics, 5) associated with changes in brain electrical activity, and 6) associated with changes in gene expression (Shaw et al., 2000; Hendricks et al., 2000; Nitz et al., 2002). Because of short generation times and low gene redundancy, the fruit fly is well suited to the rapid screening of sleep phenotypes. The present invention has been applied to identify Drosophila lines that i) are short sleepers; ii) do not show a sleep rebound after sleep deprivation (SD) and iii) display resistance to sleep deprivation in terms of vigilance (i.e., task performance).

The inventors have screened ˜5000 mutant lines, many of them carrying a mutation in one single gene. The mutation was caused either by the insertion of a transposon in the fly genome (insertional mutagenesis; ˜3000 lines screened so far), or by ethyl methanesulfonate (EMS, chemical mutagenesis; ˜2000 lines screened so far). Insertional lines such as those available from public stock centers, e.g. the ˜1000 lines of the Berkeley Drosophila Genome Project primary collection and the ˜2300 lines of the Rorth collection include both loss-of-function mutations and gain-of-function mutations. The first are often due to the insertion of a transposon inside a transcription unit, the latter to gene overexpression following the transposon insertion upstream of the transcription start site. Insertional and chemical mutagenesis offer different advantages. Insertional mutagenesis usually allows rapid identification of the mutated gene by sequencing the flanking sequences from one or both ends of the transposon insertion. Moreover, the mobilization of the inserted element can generate new alleles, and expression patterns can be characterized by lacZ staining of tissues. However, transposons do not insert at random into the genome, but have preferred hot spots. Chemical mutagenesis with EMS, on the other hand, randomly induces small (point) mutations over the entire genome at a reasonable rate, but the molecular characterization of the gene of interest may be not as straightforward.

In the current mutagenesis screening, mutant flies are continuously recorded in a DAMS monitor for one week, including 2-3 baseline days, 24 hours of sleep deprivation, and 1-3 days of recovery after sleep deprivation. Ten to sixteen flies (4-7 day old at the beginning of the experiment) are tested for each line. This relatively high number of flies is needed because sleep pattern and sleep amount, although consistent across different days in each individual adult fly, may vary among different flies (FIG. 3A). Interestingly, the analysis of thousand of lines has confirmed a significant difference between male and female flies: while female flies sleep almost exclusively during the night, males show also a long period of sleep in the middle of the day. The daily amount of sleep in the mutant lines tested so far shows a linear distribution, with female flies for most lines sleeping between 400 and 800 min/day, with a mean of ˜600 min, similar to that of wild-type flies (Canton-S female flies=664±137, mean ±SD; FIG. 3B). Ten lines have so far qualified as “short-sleepers”, i.e. their daily sleep amount is less than 2 standard deviations from the mean of all mutant lines tested so far (<280 min/day in female flies; FIG. 3B). An example of a short sleeper line is shown in FIG. 3C.

One of the short sleeper lines, EP(2)2162, carries a transposon in the gene encoding the regulatory (inhibitory) subunit of protein kinase A (PKA). Mobilization of the transposon in 6 independent revertant lines abolished the short sleeper phenotype, demonstrating that the PKA gene is responsible for the reduced amount of sleep in the EP(2)2162 line.

Almost all mutant lines tested so far showed an increase in sleep duration and a decrease in sleep fragmentation after 24 hours of sleep deprivation. As in wild-type flies, the sleep rebound is most pronounced during the first 4-6 hours immediately after the end of the sleep deprivation period, and in most cases does not persist the second day after sleep loss (FIGS. 2A and 4). Similarly to wild-type flies, most mutant lines only recover a small fraction (10-40%) of the sleep lost.

As shown in FIG. 4, the inventors have also identified 4 lines, one of which is also a short sleeper line, which show no sleep rebound after 24 hours of sleep deprivation, suggesting that this phenotype might be even more rare than the short-sleeper phenotype. Since sleep deprivation, as well as chronic sleep restriction, affects vigilance, short sleeper lines and “no-rebound” lines are particularly useful for analysis of waking performance (i.e., vigilance).

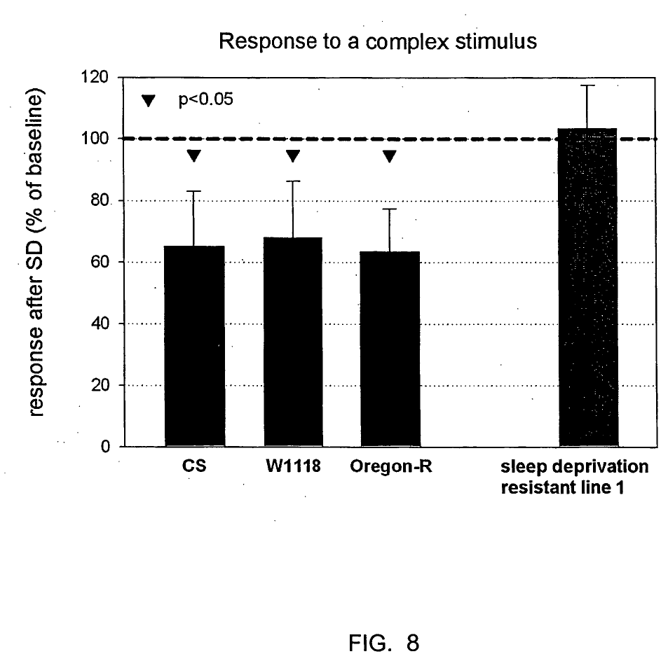

Vigilance is estimated by measuring the escape response triggered by a complex stimulus or by heat. Most wild-type flies are impaired in their vigilance after sleep deprivation. However, several mutant lines that we have isolated do not show a decrease in vigilance after sleep deprivation (see FIGS. 8-13).

In this regard, the inventors have measured the escape response elicited by exposing the mutant flies to a complex stimulus consisting of a combination of noise and vibration. Flies remained inside a DAMS monitor during the assay. The stimulus was produced by a flap vigorously pushed against the glass tubes housing the flies, and was delivered via a computer every hour at either side of the tubes. The mean latency (in sec) to crossing the infrared beam after the stimulus was delivered was measured and compared with the latency when the stimulus was not present. Flies were tested for a total of 48 hours, including one baseline day and the first recovery day after SD.

Escape responses were also measured after flies were exposed to a thermal stimulus. For this test, single flies were removed from the glass tubes and placed inside a heat box as shown in FIG. 5. The heat box includes 16 heating chambers, each housing a single fly. Inside each chamber, position and movements of the fly were recorded and displayed on line. Flies were first adapted to the chamber for at least 30 min. Temperature on either side of the chamber was then alternately increased by 4° C. every min from 22° C. (baseline value) to 44° C. The latency to crossing the infrared beam, i.e. the time a fly needed to move to the cooler side of the chamber, was measured for each temperature step. Latencies for all temperature steps were averaged for each fly. Flies were tested during the first 2 hours after the end of SD and at the same circadian time during baseline.

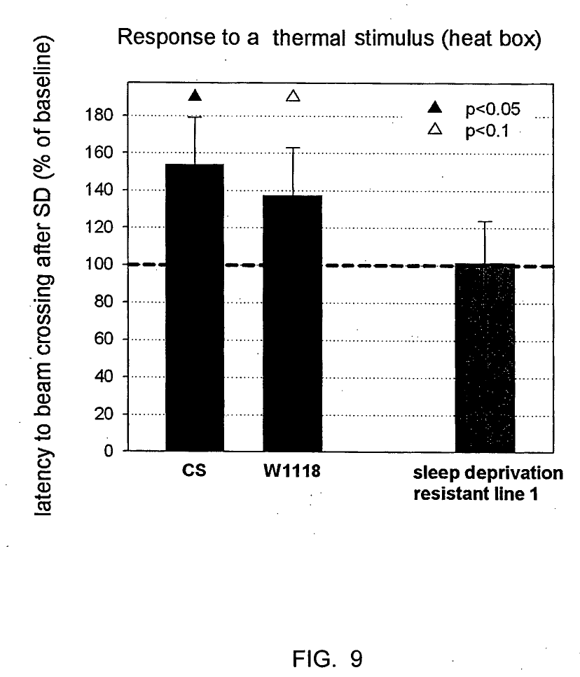

FIG. 8 illustrates the effects of sleep deprivation (SD) on the ability of flies to respond to a complex stimulus. After 24 hours of SD, all wild-type flies (CS, White 1118 and Oregon R lines) show a reduced ability to respond to a complex stimulus, while the mutant line called “SD resistant 1” is not affected. The ability of flies to respond to the complex stimulus is used as a measure of vigilance. FIG. 9 depicts the effects of sleep deprivation (SD) on the ability of flies to respond to a thermal stimulus. After 24 hours of SD, all wild-type flies (CS, White 1118 and Oregon R lines) show a reduced ability to respond to a thermal stimulus, i.e. their latency to beam crossing is increased. The latency does not change in the mutant line called “SD resistant 1”. The ability of flies to respond to the thermal stimulus is also used as a measure of vigilance.

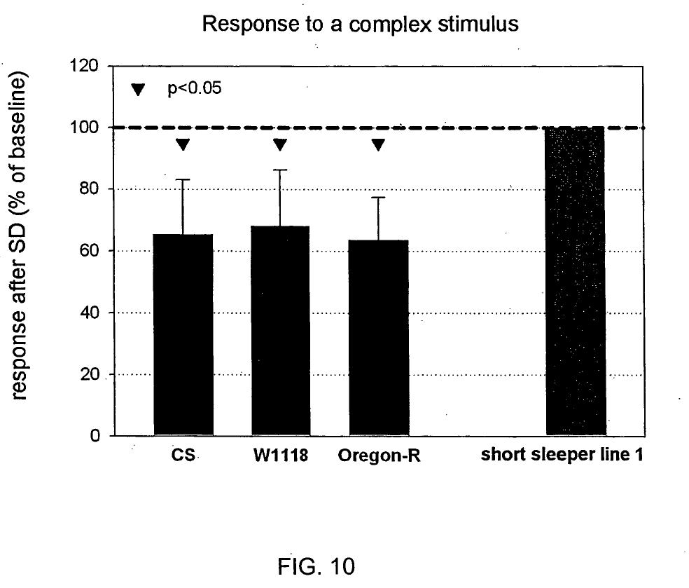

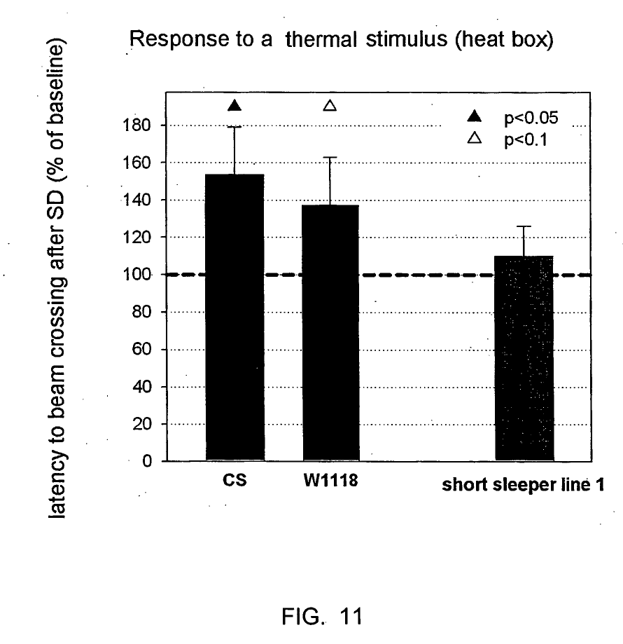

FIG. 10 shows the effects of sleep deprivation (SD) on the ability of flies to respond to a complex stimulus. After 24 hours of SD, all wild-type flies (CS, White 1118 and Oregon R lines) show a reduced ability to respond to a complex stimulus, while the mutant line called “Short Sleeper 1” is not affected. The ability of flies to respond to the complex stimulus is used as a measure of vigilance. FIG. 11 further depicts the effects of sleep deprivation (SD) on the ability of flies to respond to a thermal stimulus. After 24 hours of SD, all wild-type flies (CS, White 1118 and Oregon R lines) show a reduced ability to respond to a thermal stimulus, i.e. their latency to beam crossing is increased. The latency does not change in the mutant line called “Short Sleeper 1”. The ability of flies to respond to the thermal stimulus is also used as a measure of vigilance.

### Example 2

### Identification of Sleep-Related Two Pore Domain K+ Channels

The inventors have recently shown that the fruit fly Drosophila melanogaster sleeps very much the same way as mammals do, and can therefore be used as a model system for the genetic investigation of sleep (Shaw et al., Science, 2000; Shaw et al., Nature, 2002; Cirelli, 2003). In the context of a large-scale effort at mutagenesis screening for sleep mutants in Drosophila, the inventors have examined lines that carry a deficiency for genes codifying for two pore domain K+ channels. Analysis of Drosophila genome indicates that the fruit fly contains 12 sequences with high homology to the mammalian two pore domain K+ channels. There are no mutant alleles for any of these genes. However, there are 11 small deficiencies spanning the regions in which these genes are located. Flies heterozygous for any one of these deficiencies (homozygous lines are lethal) were tested for their sleep phenotype. As shown in Table I below, one deficiency line (stock # 5707) slept significantly less than wild-type flies (normal sleep in wild-type flies, as well as in more than 3000 mutant lines already tested, is 664±137 min/day).

TABLE 1
Drosophila genes coding for two pore domain K+ channels

| | | | | | sleep |
|---|---|---|---|---|---|
| | | | | stock | (min/ |
| Gene | map | related def | breakpoints | # | 24 h) |
| Ork1 | 9F8-12 | Df(I)vN124B | (9E3-F3; IOAI-8) | 5707 | 375 |
| CG10864 | 91A2-3 | Df(3R)P14 | (90C2; 91BI-2) | 3010 | 629 |
| CG15655 | 57B20-C1 | Df(2R)PI13 | (57B 13-14; | 1916 | 900 |
| | | | 57D8-9) | | |
| CG1756 | 1OB15-C1 | Df(1)N71 | (IOB5; IOD4) | 958 | 772 |
| CG6952 | 4CI6-DI | Df(I)RC40 | (4BI; 4FI) | 943 | 755 |
| CG3367 | 6AI-2 | Df(I)dx81 | (5C3-10; | 5281 | 743 |
| | | | 6C3-12) | | |
| CG9194 | 61F4 | Df(3L)Arl4-8 | (61C4; 62A8) | 439 | 848 |
| CG9361 | 8SD18-20 | Df(3R)by416 | (85DIO-12; | 1932 | 776 |
| | | | 85EI-2) | | |
| CG9637 | 87F7-8 | Df(3R)126c | (87EI; 87FI1) | 3009 | 581 |
| CG8713 | 44A4 | Df(2R)CA53 | (43E7-18; 44B6) | 3364 | 1065 |
| BCDNA: | 46B1-7 | Df(2R)eve 1.18 | (44B; 46D-E; | 6263 | 507 |
| GH04802 | | | 6I-100) | | |

These results confirm the inventors' observations that carrying a deficiency per se, in the X chromosome or in any other chromosome, does not cause a decrease in sleep. Moreover, the finding that the loss of one of the two copies of a gene coding for a two pore domain K+ channel is sufficient to significantly decrease sleep amount in 2 cases (see below) support the notion that some of these channels play a key role in the regulation of sleep.

Line 5707 contains a deletion (Df(I)vNI24B; breakpoints 9E3-F3; IOAI-8) spanning a portion of the X chromosome that includes the gene coding for the two pore domain K+ channel ORK1. As a first step to verify whether the absence of ORK1 is the determining factor in causing the short sleeper phenotype in line 5707, the inventors have tested other deficiency lines in the same chromosomal region. The expectation was that all the lines carrying a deletion that spans Ork1 should show a short sleeper phenotype, while those not spanning Ork1 should show a normal amount of sleep. Table 2 below indicates that this was indeed the case.

TABLE 2

| | | | sleep |
|---|---|---|---|
| Deficiency line | map | stock n. | (min/24 h) |
| Spanning Ork1 | | | |
| Df(I)vNI24B | (9F4-9FI3) | 5707 | 375 |
| Df(I)v-LII | (9C4-IOA2) | 1952 | 431 |
| Df(I)HC133 | (9B9-9F4) | 955 | 352 |
| Not spanning Orkl | | | |
| Df(l)v-LI | (9FI3-IOA5) | 6219 | 641 |
| Df(I)v-L2 | (9FI3-IOAI) | 904 | 684 |

As a second indication of the role of ORK1 in determining the short sleep phenotype, the inventors have searched the fly database to determine whether other genes in the same region have functional properties that could explain the sleep phenotype. The region of interest contains 17 genes, of which only 2 are functionally well characterized, sbr and v (see Table 3).

TABLE 3

| Gene | map | function |
|---|---|---|
| sbr | 9F4-F5ni | RNA/nucleus export |
| CG 11207 | 9F5 | ? |
| CG1655 | 9F5 | ? |
| CG17335 | 9F5 | ? |
| Orkl | 9F8-Fll | K channel |
| CG1637 | 9F8 | acid phosphatase |
| CG1582 | 9FI2 | helicase |
| CG17333 | 9F5 | gluconolactonase |
| CG15209 | 9F5 | ? |
| CG15210 | 9F5 | ? |
| CG2202 | 9F5 | transcription factor |
| CG2186 | 9F7 | ? |
| CG2157 | 9F8 | ? |
| CG 11203 | 9FI3 | ? |
| CG15208 | 9FI2 | ? |
| CG15207 | 9FI3 | ? |
| CG15206 | 9FI3 | ? |
| v | 9Fl3-IOAI | enzyme |

The inventors also studied the sleep phenotype in mutant alleles for sbr and v and found that these lines show a normal amount of sleep (Table 4).

TABLE 4

| | | | sleep |
|---|---|---|---|
| Gene | map | stock n. | (min/24 h) |
| Sbr | (9F4-F5) | 5649 | 756 |
| | | (sbrl2) | |
| sesb | (9E7) | 4687 | 624 |

In addition, vigilance of the mutant line 5707 following sleep deprivation has been assayed as compared to wild type flies. The methodology for measuring a complex stimulus and a thermal stimulus was as described in Example 1, above.

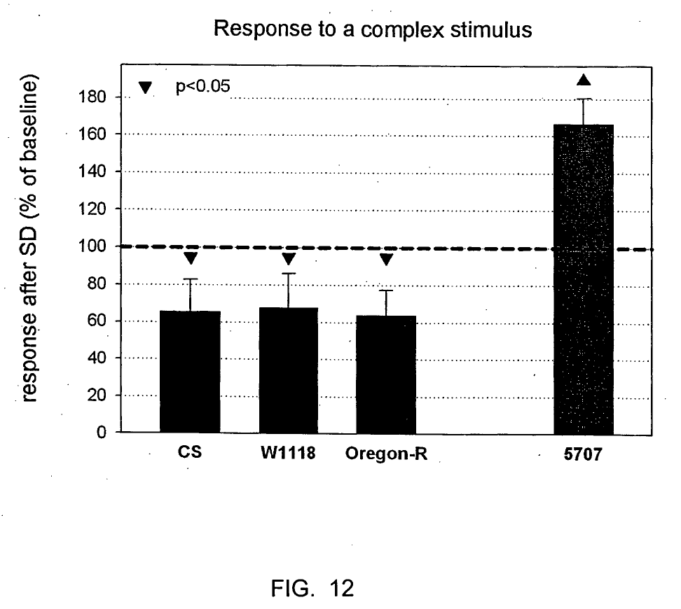

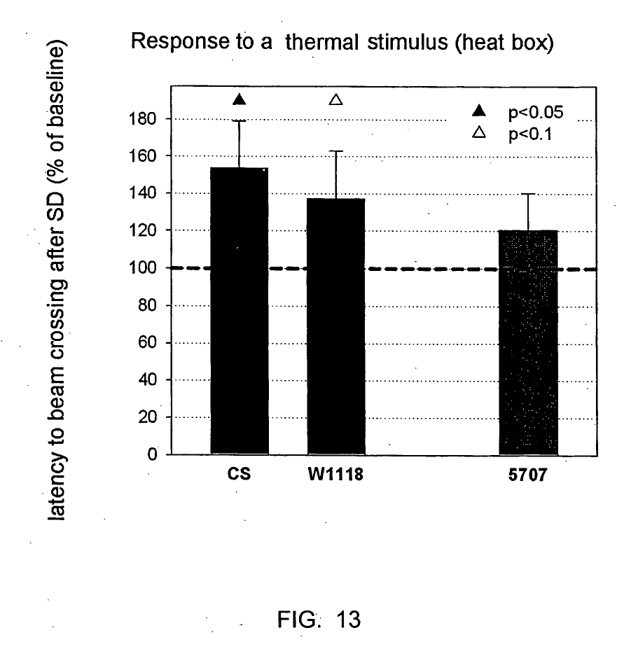

FIG. 12 depicts the effects of sleep deprivation (SD) on the ability of mutant line 5707 flies to respond to a complex stimulus following sleep deprivation. After 24 hours of SD, all wild-type flies (CS, White 1118 and Oregon R lines) show a reduced ability to respond to a complex stimulus, while the mutant line 5707, in which one of the two copies of the gene Ork1 is missing, is not affected. The ability of flies to respond to the complex stimulus is used as a measure of vigilance. FIG. 13 additionally shows the effects of sleep deprivation (SD) on the ability of flies to respond to a thermal stimulus. After 24 hours of SD, all wild-type flies (CS, White 1118 and Oregon R lines) show a reduced ability to respond to a thermal stimulus, i.e. their latency to beam crossing is increased. The latency does not change in the mutant line 5707 indicating an increased vigilance (i.e., ability to perform complex tasks).

In summary, line 5707 flies, which are heterozygous for a deletion of a small portion of the X chromosome that includes the gene coding for the two pore domain K channel ORK1, sleep much less than wild-type flies and thousands of other mutant lines. Other deficiency lines spanning the same region and including Ork1 are also short sleepers. By contrast, deficiency lines in the same region but not spanning Ork1 show a normal amount of sleep. Mutations in 2 of the 17 genes included in the deletion carried by line 5707 do not produce a short sleeper phenotype. In addition, line 5707 flies exhibit an improved vigilance following sleep deprivation as compared to wild type flies. This is a key result and provides data to support the use of two pore domain K+ channel-based assays for screening sleep-related drugs, particularly wakefulness-promoting and continuous performance drugs. Altogether, these results are consistent with the hypothesis that a specific two pore domain K+ channel plays a key role in determining the amount of sleep and, most notably, the level of vigilance exhibited by sleep-deprived subjects.

### Example 3

### Gene Expression Screening in Rats

The inventors' laboratory has pioneered the study of gene expression changes that occur in relation to sleep and waking (Cirelli et al., 1996; Cirelli and Tononi, 1998). They have recently completed a genome-wide analysis of the rat brain mRNAs using Affymetrix GeneChips RGU34 A, B, C, which include most of the known genes and expressed sequence tags. Brain gene expression was compared between rats that had been i) asleep for the first 8 h of the light period, ii) spontaneously awake for the first 8 h of the dark period, and iii) sleep deprived during the light period for 8 h. This experimental paradigm allowed the inventors to distinguish between changes in gene expression related to sleep and waking per se as opposed to changes related to circadian time or the sleep deprivation procedure.

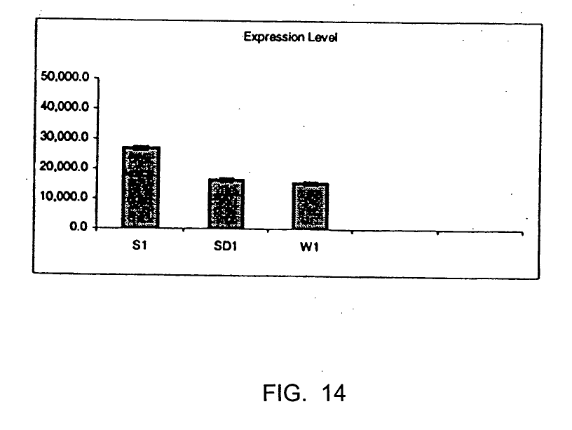

Using a highly conservative approach and systematic confirmation, the inventors found that ˜5% of the genes expressed in the cerebral cortex are up- or down-regulated between sleep and wakefulness. Of the transcripts up-regulated during spontaneous or forced wakefulness, several correspond to those the inventors had previously identified using different techniques. For the first time, functional categories of transcripts that are up-regulated during sleep have been identified. Most notable among them is a transcript corresponding to TREK-1, the gene coding the two pore domain K+ channel that the inventors had hypothesized, a priori, to be a most promising candidate to mediate sleep. The gene array results were confirmed using quantitative PCR, as indicated in FIG. 14. In FIG. 14, values on the Y axis refer to the number of mRNA copies. The X axis shows the experimental groups: S1: rats that have been spontaneously asleep for 8 hours; SDI: rats sleep deprived for 8 hours; WI: rats spontaneously awake for 8 hours. Note that transcripts levels of TREK-1 increase almost 2-fold in sleeping animals relative to awake animals.

Since increased levels of two pore domain K+ channels should increase the probability of sleep, these data are fully consistent with the notion that such channels play a key role in the genesis of sleep states. Most importantly, these data provide a direct demonstration that a specific two pore domain K+ channel, TREK-1 is involved in the regulation of sleep.

It is understood that the examples and embodiments described herein are for illustrative purposes only and that various modifications or changes in light thereof will be suggested to persons skilled in the art and are to be included within the spirit and purview of this application and scope of the appended claims. All publications, sequence submissions, patents, and patent applications cited herein are hereby incorporated by reference in their entirety for all purposes.

References

Adams M D et al. The genome sequence of Drosophila melanogaster. Science 287:2185-95, 2000.

Biesiadecki M., Huber R., Holladay C., Hill S., Tononi G., Cirelli C. Steep homeostasis in the fruit fly. Sleep 26(S): A22, 2003.

Bushnell P J. Behavioral approaches to the assessment of attention in animals. Psychopharmacol 138: 231-259, 1998.

Cirelli C, Pompeiano M, Tononi G. Neuronal gene expression in the waking state: a role for the locus coeruleus. Science 274: 1211-1215, 1996.

Cirelli C, Tononi G. Differences in gene expression between sleep and waking as revealed by mRNA differential display. Molecular Brain Research 56: 293-305, 1998.

Cirelli C, Tononi G. Differential expression of plasticity-related genes in waking and sleep and their regulation by the noradrenergic system. J. Neuroscience, 20: 9187-9194, 2000a

Cirelli C, Tononi G. Gene expression in the brain across the sleep-waking cycle. Brain Research 885: 303-321, 2000b.

Cirelli C, Hill S, Holladay C, Biesiadecki M, Martinez-Gonzalez D., Kreber R., Ganetzky B., and Tononi G. Sleep in Drosophila melanogaster: a mutagenesis screening. Sleep 26: A416, 2003.

Cirelli, C. Searching for sleep mutants of Drosophila melanogaster. BioEssays 25: 940-949, 2003.

Dinges D F, Pack F, Williams K, Gillen K A, Powell J W, Ott G E, Aptowicz C, Pack A I. Cumulative sleepiness, mood disturbance, and psychomotor vigilance performance decrements during a week of sleep restricted to 4-5 hours per night. Sleep 20:267-277, 1997.

Doran S M, Van Dongen H P A, Dinges D F. Sustained attention performance during sleep deprivation: evidence of state instability. Arch Ital Biol 139: 253-267, 2001.

Faulhaber J, Steiger A, Lancel M. The GABAA agonist THIP produces slow wave sleep and reduces spindling activity in NREM sleep in humans. Psychopharmacology 130:285-91, 1997.

Greenspan R J, Tononi G, Cirelli C, Shaw P J. Sleep and the fruit fly. Trends in Neuroscience 24: 142-145, 2001.

Harrison Y, Home J A. The impact of sleep deprivation on decision making: a review. J Exp Psychol Appl 6:236-49, 2000.

Hendricks J C, Finn S M, Panckeri K A, Chavkin J, Williams J A, Sehgal A, Pack A I. Rest in Drosophila is a sleep-like state. Neuron 25:129-38, 2000.

Huber R, Deboer T, Tobler I. Effects of sleep deprivation on sleep and sleep EEG in three mouse strains: empirical data and simulations. Brain Res. 857:8-19, 2000.

Lancel M, Wetter T C, Steiger A, Mathias S. Effect of the GABAA agonist gaboxadol on nocturnal sleep and hormone secretion in healthy elderly subjects. Am J Physiol Endocrinol Metab. 281:E130-7, 2001.

Littleton J T, Ganetzky B. Ion channels and synaptic organization: analysis of the Drosophila genome. Neuron 26:35-43, 2000.

Miklos G L, Maleszka R. Deus ex genomix. Nature Neuroscience 3:424-425, 2000.

Nitz D A, van Swinderen B, Tononi G, Greenspan R J. Local field potential recordings from Drosophila mushroom bodies and their modulation by activity state. Sleep 24S:40, 2001.

Nitz D A, van Swinderen B, Tononi G, Greenspan R J. Electrophysiological Correlates of Rest and Activity in Drosophila melanogaster. Curr. Biol. 12:1934-40, 2002.

Patel, A J, Honore, E. Properties and modulation of mammalian 2P domain K+ channels. Trends Neurosci. 24: 339-346, 2001.

Putz G., Heisenberg M. Memories in Drosophila heat-box learning. Learn Mem. 9:349-59, 2002.

Roberts D B (Ed). Drosophila. A Practical Approach. Second Edition, Oxford University Press, 1998.

Rörth P, Szabo K, Bailey A, Laverty T, Rehm J, Rubin G M, Weigmann K, Milan M, Benes V, Ansorge W, Cohen S M. Systematic gain-of-function genetics in Drosophila. Development. 125:1049-57, 1998.

Rubin G M et al. Comparative genomics of the eukaryotes. Science 287:2204-15, 2000.

Sarter M, McGaughy J. Assessment of sustained and divided attention in rats: aspects of validity, Psychopharmacol. 138;260-262, 1998.

Shaw P J, Cirelli C, Greenspan R J, Tononi G. Correlates of sleep and waking in Drosophila melanogaster. Science 287: 1834-1837, 2000a.

Shaw P J, Cirelli C, Greenspan R J, Tononi G. Genetic screening of sleep-like behavior in Drosophila melanogaster. Soc Neurosci Abstr 26: 1756, 2000b.

Spradling A C, Stem D, Beaton A, Rhem E J, Laverty T, Mozden N, Misra S, Rubin G M. The Berkeley Drosophila Genome Project gene disruption project: Single P-element insertions mutating 25% of vital Drosophila genes. Genetics 153:135-77, 1999.

Steriade M, Timofeev I, Grenier F. Natural waking and sleep states: a view from inside neocortical neurons. J Neurophysiol 85:1969-85, 2001a.

Steriade M. Impact of network activities on neuronal properties in corticothalamic systems. J Neurophysiol 86:1-39, 2001b.

Stem M, Ganetzky B. Identification and characterization of inebriated, a gene affecting neuronal excitability in Drosophila. J Neurogenet. 8:157-72, 1992.

Trudeau M C, Warmke J W, Ganetzky B, Robertson G A. HERG, a human inward rectifier in the voltage-gated potassium channel family. Science 269:92-5, 1995.

Van Dongen H P, Maislin G, Mullington J M, Dinges D F. The cumulative cost of additional wakefulness: dose-response effects on neurobehavioral functions and sleep physiology from chronic sleep restriction and total sleep deprivation. Sleep 26: 117-126, 2003.

Waddell S, Quinn W G. Flies, genes, and learning. Ann Rev Neurosci 24:1283-1309, 2001.

What is claimed is:

1. A method for identifying a Drosophila mutant fly with a no rebound phenotype, comprising the steps of:
(a) obtaining a Drosophila mutant fly;
(b) recording sleep quantity of said mutant fly over a time course including a baseline period, a sleep deprivation period, and a recovery period; and
(c) determining the effect of the sleep deprivation period on the sleep quantity during the recovery period in comparison to the sleep quantity for the baseline period wherein a no rebound Drosophila mutant fly requires significantly less sleep during the recovery period than wild type Drosophila subjected to the same time course.

2. A method according to claim 1 wherein step (b) is carried out in an automatic infrared Drosophila activity monitoring system.

3. A method according to claim 1 wherein the baseline period, sleep deprivation period and recovery period are each 24 hours in length and the no rebound mutant fly during the recovery period immediately after sleep deprivation requires, on average, less than an approximately 3.0% higher sleep quantity than during its baseline period, compared to wild type Drosophila showing, on average, a required increase of approximately 25% for the same time course.

4. A method for identifying a Drosophila mutant fly with a sleep deprivation/resistant phenotype, comprising the steps of:
(a) obtaining a Drosophila mutant fly;
(b) recording sleep quantity and vigilance of said mutant fly over a time course including a baseline period, a sleep deprivation period, and a recovery period; and
(c) determining the effect of the sleep deprivation period on the sleep quantity and vigilance during the recovery period in comparison to the sleep quantity and vigilance for the baseline period wherein a sleep deprivation resistant Drosophila mutant fly: (i) requires significantly less sleep during the recovery period than wild type Drosophila subjected to the same time course; and (ii) displays vigilance during the recovery period comparable to vigilance during the mutant fly's baseline period.

5. A method according to claim 4 wherein step (b) is carried out in an automatic infrared Drosophila activity monitoring system.

6. A method according to claim 4 wherein the baseline period, sleep deprivation period and recovery period are each 24 hours in length and the sleep deprivation resistant mutant fly during the recovery period immediately after sleep deprivation requires, on average, less than an approximately 3.0% higher sleep quantity than during its baseline period, compared to wild type Drosophila showing, on average, a required increase of approximately 25% for the same time course.

7. A method for identifying a Drosophila mutant fly with a short sleep phenotype, comprising the steps of:
(a) obtaining a mutant line of Drosophila;
(b) recording sleep quantity of said mutant line over a time course including a baseline period wherein a Drosophila mutant fly with a short sleep phenotype requires significantly less sleep during the baseline period compared to wild type Drosophila subjected to the same time course.

8. A method according to claim 7 wherein step (b) is carried out in an automatic infrared Drosophila activity monitoring system.

9. A method according to claim 7 wherein the baseline period is 24 hours in length and the mutant fly with the short sleep phenotype requires, on average, no more than 375 minutes over the baseline period.

10. A method for identifying a compound useful for promoting wakefulness in an organism requiring sleep, comprising the step of determining the effect of a test compound on a two pore domain K+ channel to thereby identify a compound which decreases the K+ current or shortens the open state of said two pore domain K+ channel wherein the decreased K+ current or shortened open state correlates with increased wakefulness in said organism.

11. A method according to claim 10 wherein said two pore domain K+ channel is TREK-1 (SEQ ID NO:2) or a homolog thereof.

12. A method according to claim 10 wherein said two pore domain K+ channel is ORK-1 (SEQ ID NO: 1) or a homolog thereof.

13. A method according to claim 10 wherein said two pore domain K+ channel is: (i) expressed in the cerebral cortex and/or thalamus of the brain; (ii) sensitive to modulation by second messengers or phosphorylation; or (iii) sensitive to general anesthetics.

14. A method according to claim 10 wherein said two pore domain K+ channel is: (i) expressed in the cerebral cortex and/or thalamus of the brain; (ii) sensitive to modulation by second messengers or phosphorylation; and (iii) sensitive to general anesthetics.

15. A method according to claim 10 wherein the method is carried out in vitro.

16. A method for identifying a compound useful for promoting highly restorative sleep in an organism requiring sleep, comprising the step of determining the effect of a test compound on a two pore domain K+ channel to thereby identify a compound which increases the K+ current or prolongs the open state of said two pore domain K+ channel wherein the increased K+ current or prolonged open state correlates with promoting highly restorative sleep in said organism.

17. A method according to claim 16 wherein said two pore domain K+ channel is TREK-1 (SEQ ID NO:2) or a homolog thereof.

18. A method according to claim 16 wherein said two pore domain K+ channel is ORK-1 (SEQ ID NO:1) or a homolog thereof.

19. A method according to claim 16 wherein said two pore domain K+ channel is: (i) expressed in the cerebral cortex and/or thalamus of the brain; (ii) sensitive to modulation by second messengers or phosphorylation; or (iii) sensitive to general anesthetics

20. A method according to claim 16 wherein said two pore domain K+ channel is: (i) expressed in the cerebral cortex and/or thalamus of the brain; (ii) sensitive to modulation by second messengers or phosphorylation; and (iii) sensitive to general anesthetics.

21. A method according to claim 16 wherein the method is carried out in vitro.

22. A method for identifying a wakefulness-promoting compound useful in providing improved wakefulness to a sleep-deprived subject, comprising the steps of:
(a) administering a test compound to wild type (wt) Drosophila; and
(b) recording sleep quantity of said wt Drosophila over a time course including a baseline period, a sleep deprivation period, and a recovery period whereby a wakefulness-promoting compound is identified as bestowing upon said wt Drosophila wakefulness wherein said wt Drosophila administered the test compound requires significantly less sleep during the sleep deprivation period than wt Drosophila not receiving the test compound and subjected to the same time course.

23. A method according to claim 22 wherein step (b) is carried out in an automatic infrared Drosophila activity monitoring system.

24. A method for identifying a continuous performance compound useful in providing improved vigilance to a sleep-deprived subject, comprising the steps of:
(a) administering a test compound to wild type (wt) Drosophila; and
(b) recording sleep quantity and vigilance of said wt Drosophila over a time course including a baseline period, a sleep deprivation period, and a recovery period; whereby a continuous performance compound is identified as bestowing upon said wt Drosophila sleep deprivation resistance wherein said wt Drosophila administered the test compound: (i) requires significantly less sleep during the recovery period than wt Drosophila not receiving the test compound and subjected to the same time course; and (ii) displays vigilance during the recovery period comparable to vigilance during the baseline period.

25. A method according to claim 24 wherein step (b) is carried out in an automatic infrared Drosophila activity monitoring system.
